(19) United States
(12) Patent Application Publication
(10) Pub. No.: US 2023/0200784 A1
(43) Pub. Date: Jun. 29, 2023

(54) ULTRASONIC DIAGNOSTIC DEVICE, IMAGE PROCESSING DEVICE, AND IMAGE PROCESSING METHOD

(71) Applicant: Canon Medical Systems Corporation, Otawara-shi, Tochigi (JP)

(72) Inventors: Yutaka KOBAYASHI, Nasushiobara (JP); Yukifumi KOBAYASHI, Yokohama (JP); Satoshi MATSUNAGA, Nasushiobara (JP); Yoshitaka MINE, Nasushiobara (JP); Atsushi NAKAI, Nasushiobara (JP); Jiro HIGUCHI, Otawara (JP); Kazuo TEZUKA, Nasushiobara (JP); Shigemitsu NAKAYA, Nasushiobara (JP)

(73) Assignee: Canon Medical Systems Corporation, Otawara-shi, Tochigi (JP)

(21) Appl. No.: 18/173,583

(22) Filed: Feb. 23, 2023

Related U.S. Application Data
(62) Division of application No. 15/864,060, filed on Jan. 8, 2018, now abandoned.

(30) Foreign Application Priority Data
Jan. 10, 2017 (JP) 2017-002058
Dec. 27, 2017 (JP) 2017-251159

(51) Int. Cl. A61B 8/08; A61B 8/00; A61B 8/06

(52) U.S. Cl. CPC A61B 8/5223; A61B 8/4254; A61B 8/54; A61B 8/06; A61B 8/483; A61B 8/145

(57) ABSTRACT

An ultrasonic diagnostic device includes an ultrasonic probe and processing circuitry. The probe conducts ultrasonic scanning on a three-dimensional area of a subject and receives a reflected wave from the subject. The circuitry acquires the correspondence relation between a position in ultrasonic image data on the three-dimensional area based on the reflected wave and a position in volume data on the subject captured by a different medical-image diagnostic device. The circuitry receives, from an operator, an operation to set a position marker, which indicates the position at which blood-flow information is extracted, on a scan area of the ultrasonic image data. The circuitry causes the image generated during a rendering process on the ultrasonic image data to be displayed and causes the position marker to be displayed at a corresponding position on a display image based on at least the volume data in accordance with the correspondence relation.

### CROSS-REFERENCE TO RELATED APPLICATIONS

This application is a divisional application of U.S. application Ser. No. 15/864,060, filed Jan. 8, 2018, which is based upon and claims the benefit of priority from Japanese Patent Application No. 2017-002058, filed on Jan. 10, 2017 and Japanese Patent Application No. 2017-251159, filed on Dec. 27, 2017; the entire contents of which are incorporated herein by reference.

### FIELD

Embodiments described herein relate generally to an ultrasonic diagnostic device, an image processing device, and an image processing method.

### BACKGROUND

Conventionally, ultrasonic diagnostic devices display the Doppler spectrum (Doppler waveform) that represents blood-flow information by using Doppler information (Doppler signals) that is extracted from reflected waves of ultrasound. The Doppler waveform is a time-series plotted waveform of a blood flow velocity at the position that is set as an observation site by an operator. For example, the operator sets the position, at which the blood-flow information is extracted, on a two-dimensional ultrasonic image (two-dimensional B-mode image or two-dimensional color Doppler image).

For example, in a Pulsed Wave Doppler (PWD) mode for collecting Doppler waveforms according to the PWD method, an operator locates a position marker, which indicates the position of a sample volume (or sampling gate) in a specific site within a blood vessel in accordance with the location of the blood vessel that is rendered on a two-dimensional ultrasonic image. In the PWD mode, the Doppler waveform, which indicates the blood-flow information in the sample volume, is displayed. Furthermore, for example, in a Continuous Wave Doppler (CWD) mode for collecting a Doppler waveform according to the CWD method, an operator locates a position marker, which indicates a linear sampling position, so as to pass the blood vessel that is rendered on a two-dimensional ultrasonic image. In the CWD mode, the Doppler waveform that indicates the entire blood-flow information on the scan line (beam line), which is set on the sampling position, is displayed.

### BRIEF DESCRIPTION OF THE DRAWINGS

FIG. 1 is a block diagram that illustrates an example of the configuration of an ultrasonic diagnostic device according to a first embodiment;

FIG. 2 is a diagram that illustrates a process of an acquisition function according to the first embodiment;

FIGS. 3A and 3B are diagrams that illustrate a process of a reception function according to the first embodiment;

FIG. 4 is a flowchart that illustrates the steps of the process of the ultrasonic diagnostic device according to the first embodiment;

FIG. 5 is a diagram that illustrates a process of the reception function according to a modified example 1 of the first embodiment;

FIG. 6 is a diagram that illustrates a process of a display control function according to a modified example 2 of the first embodiment;

FIG. 7 is a diagram that illustrates a process of the display control function according to a second embodiment;

FIG. 8 is a diagram that illustrates a process of the display control function according to the second embodiment;

FIG. 9 is a diagram that illustrates a process of the display control function according to a third embodiment;

FIG. 10 is a block diagram that illustrates an example of the configuration of the ultrasonic diagnostic device according to a fourth embodiment;

FIG. 11 is a diagram that illustrates a process of the display control function according to the fourth embodiment;

FIG. 12 is a block diagram that illustrates an example of the configuration of the ultrasonic diagnostic device according to a fifth embodiment;

FIGS. 13A and 13B are diagrams that illustrate a process of the reception function according to the fifth embodiment;

FIG. 14 is a diagram that illustrates a process of the ultrasonic diagnostic device according to a sixth embodiment;

FIG. 15 is a diagram that illustrates a process of the display control function according to a different embodiment; and

FIG. 16 is a diagram that illustrates a process of the display control function according to a different embodiment.

### DETAILED DESCRIPTION

The problem solved by embodiments is to provide an ultrasonic diagnostic device, an image processing device, and an image processing method, with which the accuracy and the quantitative characteristic of blood-flow information may be improved.

An ultrasonic diagnostic device according to an embodiment includes an ultrasonic probe and processing circuitry. The ultrasonic probe conducts ultrasonic scanning on a three-dimensional area of a subject and receives a reflected wave from the subject. The processing circuitry acquires the correspondence relation between a position in ultrasonic image data on the three-dimensional area based on the reflected wave and a position in volume data on the subject captured by a different medical-image diagnostic device. The processing circuitry receives, from an operator, an operation to set a position marker, which indicates the position at which blood-flow information is extracted, on a scan area of the ultrasonic image data. The processing circuitry causes the image generated during a rendering process on the ultrasonic image data to be displayed and causes the position marker to be displayed at a corresponding position on a display image based on at least the volume data in accordance with the correspondence relation.

With reference to the drawings, an explanation is given below of an ultrasonic diagnostic device, an image processing device, and an image processing method according to embodiments. Furthermore, the embodiments described below are examples, and the ultrasonic diagnostic device, the image processing device, and the image processing method according to the embodiments are not limited to the following explanations.

### First Embodiment

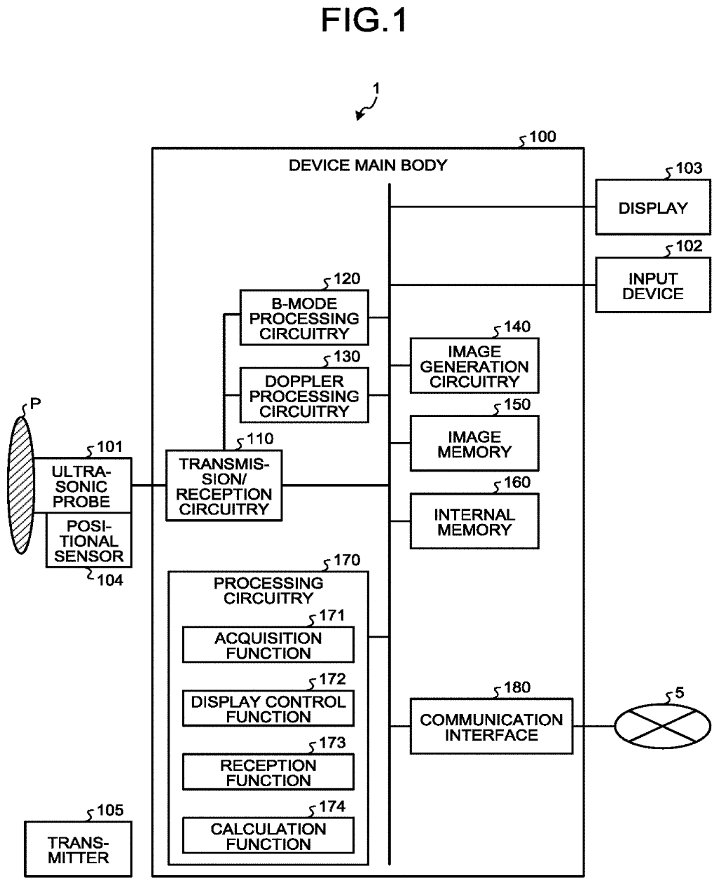

FIG. 1 is a block diagram that illustrates an example of the configuration of an ultrasonic diagnostic device 1 according to a first embodiment. As illustrated in FIG. 1, the ultrasonic diagnostic device 1 according to the first embodiment includes a device main body 100, an ultrasonic probe 101, an input device 102, a display 103, a positional sensor 104, and a transmitter 105. The ultrasonic probe 101, the input device 102, the display 103, and the transmitter 105 are communicatively connected to the device main body 100.

The ultrasonic probe 101 includes multiple piezoelectric vibrators, and the piezoelectric vibrators generate ultrasonic waves in accordance with drive signals that are fed from transmission/reception circuitry 110 included in the device main body 100. Furthermore, the ultrasonic probe 101 receives reflected waves from a subject P and converts them into electric signals. Specifically, the ultrasonic probe 101 conducts ultrasonic scanning on the subject P to receive reflected waves from the subject P. Furthermore, the ultrasonic probe 101 includes a matching layer that is provided in the piezoelectric vibrator, a backing member that prevents propagation of ultrasonic waves backward from the piezoelectric vibrator, or the like. Furthermore, the ultrasonic probe 101 is connected to the device main body 100 in an attachable and removable manner.

After ultrasonic waves are transmitted from the ultrasonic probe 101 to the subject P, the transmitted ultrasonic waves are sequentially reflected by discontinuous surfaces of the acoustic impedance in the body tissues of the subject P, and they are received as reflected-wave signals by the piezoelectric vibrators included in the ultrasonic probe 101. The amplitude of the received reflected-wave signal depends on the difference in the acoustic impedance on the discontinuous surfaces, which reflect ultrasonic waves. Furthermore, in a case where transmitted ultrasonic pulses are reflected by surfaces of moving blood flows, the heart wall, or the like, reflected-wave signals are subjected to frequency shift due to the Doppler effect by being dependent on the velocity component in an ultrasonic transmission direction of a movable body.

The first embodiment uses the ultrasonic probe 101 that conducts two-dimensional scanning on the subject P by using ultrasonic waves. For example, the ultrasonic probe 101 is a 1D array probe on which multiple piezoelectric vibrators are arranged in one column. The 1D array probe is, for example, a sector-type ultrasonic probe, a linear-type ultrasonic probe, or a convex-type ultrasonic probe. Furthermore, according to the first embodiment, the ultrasonic probe 101 may be, for example, a mechanical 4D probe or a 2D array probe that is capable of conducting three-dimensional scanning on the subject P as well as two-dimensional scanning on the subject P by using ultrasonic waves. The mechanical 4D probe is capable of conducting two-dimensional scanning by using multiple piezoelectric vibrators, arranged in one column, and is also capable of conducting three-dimensional scanning by oscillating multiple piezoelectric vibrators, arranged in one column, at a predetermined angle (oscillation angle). Furthermore, the 2D array probe is capable of conducting three-dimensional scanning by using multiple piezoelectric vibrators arranged in a matrix and is also capable of conducting two-dimensional scanning by transmitting and receiving ultrasonic waves through convergence. Furthermore, the 2D array probe is capable of simultaneously conducting two-dimensional scanning on multiple cross-sectional surfaces.

Furthermore, as described below, the ultrasonic diagnostic device 1 according to the present embodiment collects Doppler waveforms by using a Pulsed Wave Doppler (PWD) method or a Continuous Wave Doppler (CWD) method. According to the present embodiment, the ultrasonic probe 101, connected to the device main body 100, is an ultrasonic probe that is capable of conducting ultrasonic-wave transmission/reception for capturing B-mode image data and color Doppler image data and ultrasonic-wave transmission/reception for collecting Doppler waveforms in a PW mode according to the PW Doppler method or in a CW mode according to the CW Doppler method.

The input device 102 includes a mouse, keyboard, button, panel switch, touch command screen, wheel, dial, foot switch, trackball, joystick, or the like, so that it receives various setting requests from an operator of the ultrasonic diagnostic device 1 and transfers the various received setting requests to the device main body 100.

The display 103 presents a graphical user interface (GUI) for an operator of the ultrasonic diagnostic device 1 to input various setting requests by using the input device 102 or presents ultrasonic image data, or the like, generated by the device main body 100. Furthermore, the display 103 presents various types of messages to notify an operator of the operation status of the device main body 100. Furthermore, the display 103 includes a speaker so that it may also output sounds. For example, the speaker of the display 103 outputs predetermined sounds, such as beep sounds, to notify an operator of the operation status of the device main body 100.

The positional sensor 104 and the transmitter 105 are devices (position detection systems) for acquiring the positional information on the ultrasonic probe 101. For example, the positional sensor 104 is a magnetic sensor that is secured to the ultrasonic probe 101. Furthermore, for example, the transmitter 105 is a device that is located in an arbitrary position and that forms a magnetic field outward from the device as a center.

The positional sensor 104 detects a three-dimensional magnetic field that is formed by the transmitter 105. Then, on the basis of the information on the detected magnetic field, the positional sensor 104 calculates the position (coordinates) and the direction (angle) of the device in the space where the transmitter 105 serves as an origin, and it transmits the calculated position and direction to processing circuitry 170 that is described later. The three-dimensional positional information (position and direction) of the positional sensor 104, transmitted to the processing circuitry 170, is used by being converted as appropriate into the positional information on the ultrasonic probe 101 or the positional information on the scan range that is scanned by the ultrasonic probe 101. For example, the positional information of the positional sensor 104 is converted into the positional information on the ultrasonic probe 101 in accordance with the positional relationship between the positional sensor 104 and the ultrasonic probe 101. Furthermore, the positional information of the ultrasonic probe 101 is converted into the positional information on the scan range in accordance with the positional relationship between the ultrasonic probe 101 and the scan range. Moreover, the positional information on the scan range may be converted into each pixel location in accordance with the positional relationship between the scan range and a sample point on the scan line. Specifically, the three-dimensional positional information of the positional sensor 104 may be converted into each pixel location of the ultrasonic image data that is captured by the ultrasonic probe 101.

Furthermore, the present embodiment is applicable to a case where the positional information on the ultrasonic probe 101 is acquired by systems other than the above-described position detection system. For example, according to the present embodiment, there may be a case where the positional information on the ultrasonic probe 101 is acquired by using a gyroscope, an acceleration sensor, or the like.

The device main body 100 is a device that generates ultrasonic image data on the basis of reflected-wave signals that are received by the ultrasonic probe 101. The device main body 100, illustrated in FIG. 1, is a device that may generate two-dimensional ultrasonic image data on the basis of the two-dimensional reflected-wave data that is received by the ultrasonic probe 101.

As illustrated in FIG. 1, the device main body 100 includes the transmission/reception circuitry 110, B-mode processing circuitry 120, Doppler processing circuitry 130, an image generation circuit 140, an image memory 150, an internal memory 160, and the processing circuitry 170. The transmission/reception circuitry 110, the B-mode processing circuitry 120, the Doppler processing circuitry 130, the image generation circuit 140, the image memory 150, the internal memory 160, and the processing circuitry 170 are communicatively connected to one another. Furthermore, the device main body 100 is connected to a network 5 within a hospital.

The transmission/reception circuitry 110 includes a pulse generator, a transmission delay unit, a pulsar, or the like, and it feeds drive signals to the ultrasonic probe 101. The pulse generator repeatedly generates rate pulses to form transmission ultrasonic waves at a predetermined rate frequency. Furthermore, the transmission delay unit converges the ultrasonic waves, generated by the ultrasonic probe 101, into a beam-like shape and gives a delay time, which is needed to determine the transmission directivity for each piezoelectric vibrator, to each rate pulse generated by the pulse generator. Moreover, the pulsar applies drive signals (drive pulses) to the ultrasonic probe 101 at timing based on the rate pulse. That is, the transmission delay unit changes a delay time, which is given to each rate pulse, to arbitrarily adjust the transmission direction of ultrasonic waves that are transmitted from a piezoelectric vibrator surface.

Furthermore, the transmission/reception circuitry 110 has a function to instantly change a transmission frequency, a transmission drive voltage, or the like, to perform a predetermined scan sequence in accordance with a command of the processing circuitry 170 that is described later. Particularly, changes in the transmission drive voltage are made by a linear-amplifier type oscillation circuit, which may instantly change the value, or a mechanism that electrically changes multiple power supply units.

Furthermore, the transmission/reception circuitry 110 includes a pre-amplifier, an analog/digital (A/D) converter, a reception delay unit, an adder, or the like, and performs various types of processing on reflected-wave signals, received by the ultrasonic probe 101, to generate reflected-wave data. The pre-amplifier amplifies reflected-wave signals for each channel. The A/D converter conducts A/D conversion on the amplified reflected-wave signals. The reception delay unit supplies a delay time that is needed to determine the reception directivity. The adder performs an add operation on the reflected-wave signals, which have been processed by the reception delay unit, to generate reflected-wave data. Due to the add operation of the adder, reflection components are emphasized in the direction that corresponds to the reception directivity of the reflected-wave signal, and the entire beam for ultrasonic wave transmission/reception is formed due to the reception directivity and the transmission directivity.

When two-dimensional scanning is conducted on the subject P, the transmission/reception circuitry 110 causes the ultrasonic probe 101 to transmit a two-dimensional ultrasonic beam. Then, the transmission/reception circuitry 110 generates two-dimensional reflected-wave data from the two-dimensional reflected-wave signals that are received by the ultrasonic probe 101. Furthermore, when three-dimensional scanning is conducted on the subject P, the transmission/reception circuitry 110 according to the present embodiment causes the ultrasonic probe 101 to transmit a three-dimensional ultrasonic beam. Then, the transmission/reception circuitry 110 generates three-dimensional reflected-wave data from the three-dimensional reflected-wave signals that are received by the ultrasonic probe 101.

Here, various forms may be selected as the form of output signals from the transmission/reception circuitry 110; in some case, they are signals that include phase information, what are called radio frequency (RF) signals, or in some case, amplitude information after an envelope detection process.

The B-mode processing circuitry 120 receives reflected-wave data from the transmission/reception circuitry 110 and performs logarithm amplification, envelope detection process, or the like, to generate data (B mode data) that represents signal intensity with the level of luminance.

The Doppler processing circuitry 130 conducts frequency analysis on the velocity information from the reflected-wave data, received from the transmission/reception circuitry 110, extracts blood flows, tissues, or contrast-agent echo components due to the Doppler effect, and generates data (Doppler data), for which movable body information, such as velocity, dispersion, or power, are extracted at many points.

Furthermore, the B-mode processing circuitry 120 and the Doppler processing circuitry 130, illustrated in FIG. 1, may process both two-dimensional reflected-wave data and three-dimensional reflected-wave data. Specifically, the B-mode processing circuitry 120 generates two-dimensional B mode data from two-dimensional reflected-wave data and generates three-dimensional B mode data from three-dimensional reflected-wave data. Furthermore, the Doppler processing circuitry 130 generates two-dimensional Doppler data from two-dimensional reflected-wave data and generates three-dimensional Doppler data from three-dimensional reflected-wave data.

The image generation circuit 140 generates ultrasonic image data from the data generated by the B-mode processing circuitry 120 and the Doppler processing circuitry 130. Specifically, the image generation circuit 140 generates two-dimensional B-mode image data, which represents the intensity of a reflected wave with luminance, from the two-dimensional B mode data that is generated by the B-mode processing circuitry 120. Furthermore, the image generation circuit 140 generates the two-dimensional Doppler image data, which represents movable body information, from the two-dimensional Doppler data generated by the Doppler processing circuitry 130.

Two-dimensional Doppler image data is a velocity image, a dispersion image, a power image, or an image that combines them. Furthermore, the image generation circuit 140 may generate M mode image data from the time-series data of B mode data on one scan line, generated by the B-mode processing circuitry 120. Furthermore, the image generation circuit 140 may generate time-series plotted Doppler waveforms of the velocity information on blood flows or tissues from the Doppler data generated by the Doppler processing circuitry 130.

Here, generally, the image generation circuit 140 converts (scan-converts) a scan-line signal sequence for ultrasonic scanning into a scan-line signal sequence for video format, typically televisions, or the like, and generates ultrasonic image data for display. Specifically, the image generation circuit 140 conducts coordinate conversion in accordance with a scanning form of ultrasonic waves by the ultrasonic probe 101, thereby generating ultrasonic image data for display. Furthermore, in addition to scan conversion, the image generation circuit 140 performs various types of image processing, such as image processing (smoothing process) to regenerate an average value image of the luminance by using multiple image frames after scan conversion, or image processing (edge enhancement process) that uses a differential filter within an image. Furthermore, the image generation circuit 140 synthesizes ultrasonic image data with textual information on various parameters, scale marks, body marks, or the like.

That is, B mode data and Doppler data are ultrasonic image data before a scan conversion process, and data generated by the image generation circuit 140 is ultrasonic image data for display after a scan conversion process. Here, the B mode data and the Doppler data are also called raw data. The image generation circuit 140 generates “two-dimensional B-mode image data or two-dimensional Doppler image data”, which is two-dimensional ultrasonic image data for display, from “two-dimensional B mode data or two-dimensional Doppler data”, which is two-dimensional ultrasonic image data before a scan conversion process.

Furthermore, the image generation circuit 140 performs a rendering process on ultrasonic volume data to generate various types of two-dimensional image data for displaying the ultrasonic volume data on the display 103. The rendering process performed by the image generation circuit 140 includes a process to generate MPR image data from ultrasonic volume data by conducting Multi Planer Reconstruction (MPR). Furthermore, the rendering process performed by the image generation circuit 140 includes a process to perform “Curved MPR” on ultrasonic volume data or a process to conduct “Maximum Intensity Projection” on ultrasonic volume data. Furthermore, the rendering process performed by the image generation circuit 140 includes a volume rendering (VR) process to generate two-dimensional image data, to which three-dimensional information is applied, and a surface rendering (SR) process.

The image memory 150 is a memory that stores image data for display, generated by the image generation circuit 140. Furthermore, the image memory 150 may store the data generated by the B-mode processing circuitry 120 or the Doppler processing circuitry 130. B mode data and Doppler data stored in the image memory 150 may be invoked by an operator after diagnosis, for example, and it becomes ultrasonic image data for display by being passed through the image generation circuit 140.

The internal memory 160 stores various types of data, such as control programs for performing ultrasonic wave transmission/reception, image processing, and display processing, diagnosis information (e.g., patient ID or doctor's observations), diagnosis protocols, or various body marks. Furthermore, the internal memory 160 is used to store image data, or the like, which is stored in the image memory 150, as needed. Furthermore, the data stored in the internal memory 160 may be transferred to an external device via an undepicted interface. Moreover, the external device is, for example, a personal computer (PC) that is used by a doctor to conduct image diagnosis, a storage medium, such as CD or DVD, or a printer.

The processing circuitry 170 performs control of the overall operation of the ultrasonic diagnostic device 1. Specifically, the processing circuitry 170 controls operations of the transmission/reception circuitry 110, the B-mode processing circuitry 120, the Doppler processing circuitry 130, and the image generation circuit 140 in accordance with various setting requests input from an operator via the input device 102 or various control programs and various types of data read from the internal memory 160. Furthermore, the processing circuitry 170 controls the display 103 so as to present the ultrasonic image data for display, stored in the image memory 150 or the internal memory 160.

A communication interface 180 is an interface for communicating with various devices within a hospital via the network 5. With the communication interface 180, the processing circuitry 170 performs communications with external devices. For example, the processing circuitry 170 receives medical image data (X-ray computed tomography (CT) image data, magnetic resonance imaging (MRI) image data, or the like) captured by a medical-image diagnostic device other than the ultrasonic diagnostic device 1, via the network 5. Then, the processing circuitry 170 causes the display 103 to present the received medical image data together with the ultrasonic image data captured by the device. Furthermore, the displayed medical image data may be an image on which image processing (rendering process) has been performed by the image generation circuit 140. Moreover, there may be a case where the medical image data displayed together with ultrasonic image data is acquired via a storage medium, such as CD-ROM, MO, or DVD.

Furthermore, the processing circuitry 170 performs an acquisition function 171, a reception function 173, a calculation function 174, and a display control function 172. Moreover, the processing details of the acquisition function 171, the reception function 173, the calculation function 174, and the display control function 172, performed by the processing circuitry 170, are described later.

Here, for example, the respective processing functions performed by the reception function 173, the calculation function 174, and the display control function 172, which are components of the processing circuitry 170 illustrated in FIG. 1, are recorded in the internal memory 160 in the form of program executable by a computer. The processing circuitry 170 is a processor that reads each program from the internal memory 160 and executes it to implement the function that corresponds to the program. In other words, the processing circuitry 170 in a state where each program has been read has each function illustrated in the processing circuitry 170 in FIG. 1.

Furthermore, in the explanation according to the present embodiment, the single processing circuitry 170 implements each processing function that is described below; however, a processing circuit is configured by combining multiple independent processors, and each processor may execute a program to implement the function.

The term “processor” used in the above explanation means, for example, a central processing unit (CPU), a graphics processing unit (GPU), or a circuit, such as an Application Specific Integrated Circuit (ASIC), a programmable logic device (e.g., a simple programmable logic device (SPLD), a complex programmable logic device (CPLD), or a field programmable gate array (FPGA)). The processor reads the program stored in the internal memory 160 and executes it, thereby implementing the function. Furthermore, instead of storing programs in the internal memory 160, a configuration may be such that programs are directly installed in a circuit of a processor. In this case, the processor reads the program installed in the circuit and executes it, thereby implementing the function. Furthermore, with regard to each processor according to the present embodiment, as well as the case where each processor is configured as a single circuit, multiple independent circuits may be combined to be configured as a single processor to implement the function. Moreover, multiple components in each figure may be integrated into a single processor to implement the function.

The overall configuration of the ultrasonic diagnostic device 1 according to the first embodiment is explained above. With this configuration, the ultrasonic diagnostic device 1 according to the first embodiment performs each of the following processing functions in order to improve the accuracy and the quantitative characteristic of blood-flow information.

With reference to the drawings, an explanation is given below of each processing function of the ultrasonic diagnostic device 1 according to the first embodiment. Furthermore, in the case described in the following explanation, for example, ultrasonic image data and the previously captured X-ray CT image data are simultaneously displayed; however, this is not a limitation on the embodiment. For example, the embodiment is applicable to a case where ultrasonic image data and MRI image data are simultaneously displayed. Furthermore, in the case described in the following explanation, for example, the embodiment is applied to collection of Doppler waveforms according to the PWD method; however, this is not a limitation on the embodiment. For example, the embodiment is applicable to collection of Doppler waveforms according to the CWD method.

The acquisition function 171 acquires the correspondence relation between a position in the ultrasonic image data based on reflected waves of the subject P and a position in the volume data on the subject P captured by a different medical-image diagnostic device. For example, the acquisition function 171 acquires the positional information on B-mode image data in a three-dimensional space from the position detection system (the positional sensor 104 and the transmitter 105). Then, the acquisition function 171 matches the positions of the two-dimensional B-mode image data and the previously captured three-dimensional X-ray CT image data. Specifically, as the correspondence relation, the acquisition function 171 generates a conversion function of the positional information on the B-mode image data in a three-dimensional space and the coordinate information on the X-ray CT image data. Here, the acquisition function 171 is an example of an acquiring unit.

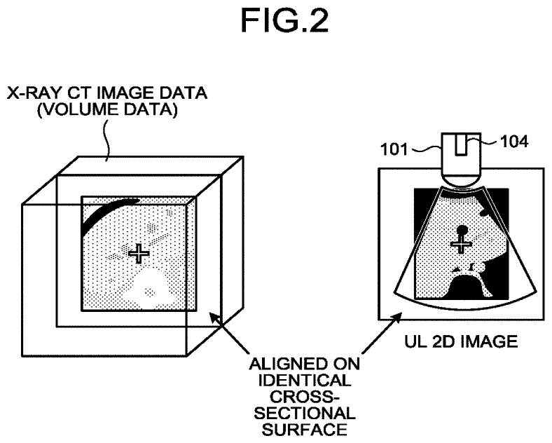

FIG. 2 is a diagram that illustrates a process of the acquisition function 171 according to the first embodiment. In FIG. 2, an explanation is given of alignment between two-dimensional B-mode image data and three-dimensional X-ray CT image data.

First, an operator makes a request to receive the previously captured X-ray CT image data on the inside of the body of the subject P from a different device. Thus, as illustrated in the left section of FIG. 2, the acquisition function 171 acquires X-ray CT image data (volume data), which is the target to be aligned. Furthermore, the operator conducts ultrasonic scanning to capture the inside of the body of the subject P, which is the target to be displayed. For example, the operator uses the ultrasonic probe 101 to conduct two-dimensional ultrasonic scanning on the subject P on a predetermined cross-sectional surface.

Then, the operator views an ultrasonic image (an UL 2D image illustrated in FIG. 2) that is presented on the display 103 while operating the ultrasonic probe 101 secured to the positional sensor 104 such that a feature site (landmark site), which serves as a mark, is rendered on the ultrasonic image. Furthermore, the operator adjusts the cross-sectional position for Multi Planar Reconstructions (MPR) processing via the input device 102 such that the cross-sectional image of the X-ray CT image data, in which the feature site is rendered, is presented on the display 103.

Then, after the same site as the feature site, rendered on the cross-sectional image of the X-ray CT image data, is rendered on the UL 2D image, the operator presses a confirmation button. Thus, the ultrasonic image presented on the display 103 temporarily freezes (remains still) and the information on each pixel location of the freezing ultrasonic image is acquired on the basis of the three-dimensional positional information of the positional sensor 104.

Then, the operator designates the center position of the feature site on each of the cross-sectional images of the fixed UL 2D image and X-ray CT image data by using for example a mouse. Thus, the acquisition function 171 determines that the feature site designated on the UL 2D image and the feature site designated on the X-ray CT image data have the same coordinates. Specifically, the acquisition function 171 specifies the coordinates of the feature site designated on the UL 2D image as the coordinates of the feature site designated on the X-ray CT image data.

In the same manner, by using a different feature site, the operator specifies the coordinates of the different feature site in the X-ray CT image data. Then, after the coordinates on the X-ray CT image data are determined with regard to multiple (3 or more) feature sites, the acquisition function 171 uses each of the determined coordinates to generate a conversion function of the positional information on the ultrasonic image data in the three-dimensional space and the coordinate information on the X-ray CT image data. Thus, for example, even if new ultrasonic image data is generated due to a shift in the position of the ultrasonic probe 101, the acquisition function 171 may relate the coordinates in the ultrasonic image data and the X-ray CT image data.

In this manner, the acquisition function 171 aligns the two-dimensional B-mode image data and the three-dimensional X-ray CT image data. Here, the explanation of the above-described acquisition function 171 is an example, and this is not a limitation. For example, the acquisition function 171 may align three-dimensional B-mode image data and three-dimensional X-ray CT image data. Furthermore, the method by which the acquisition function 171 adjusts a position is not limited to the above-described method and, for example, a known technology, such as alignment that uses a cross-correlation technique, may be used for implementation.

The display control function 172 causes the B-mode image (cross-sectional image), which corresponds to the scan cross-sectional surface on which ultrasonic scanning is conducted, to be displayed and causes the cross-sectional image of the X-ray CT image data at the position that corresponds to the B-mode image to be displayed. For example, the display control function 172 uses the conversion function, generated by the acquisition function 171, to determine the cross-sectional position that is in the X-ray CT image data and that corresponds to the cross-sectional surface of the B-mode image. Then, the display control function 172 generates two-dimensional image data (also referred to as “2D CT image”), which corresponds to the determined cross-sectional position, through MPR processing and presents it on the display 103.

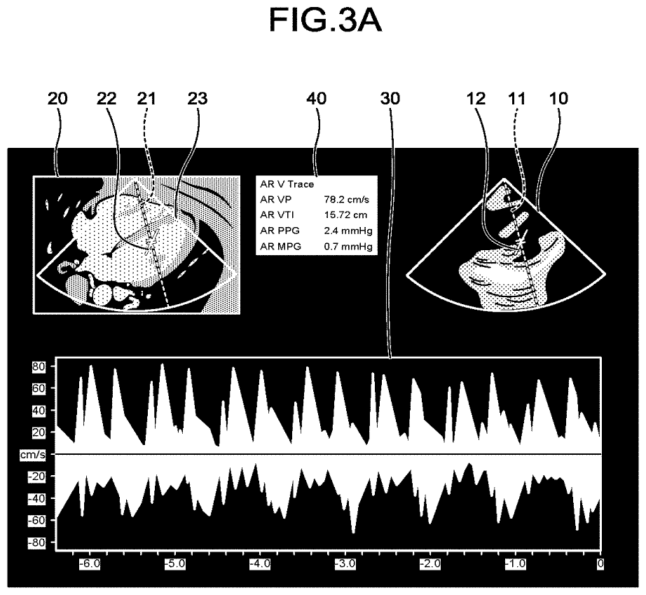

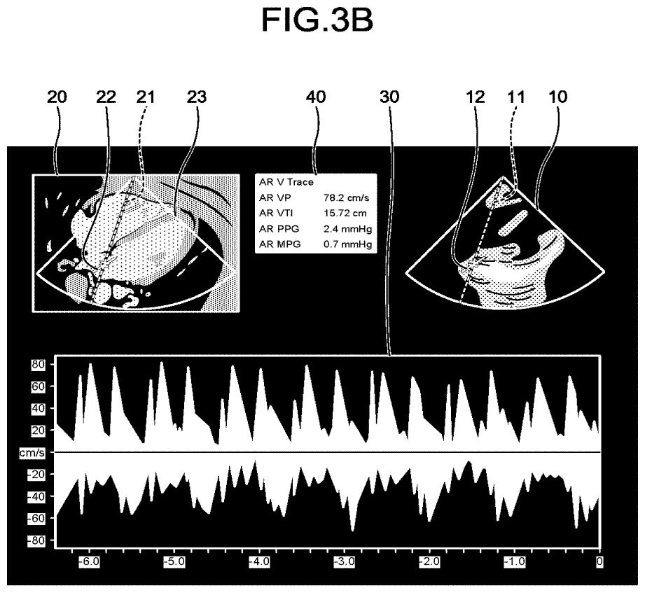

Furthermore, in accordance with the correspondence relation, the display control function 172 causes a range gate marker to be displayed at a corresponding position on the display image based on at least the X-ray CT image data. For example, the display control function 172 causes a range gate marker, which indicates the position of a sample volume, to be displayed on an ultrasonic image and a 2D CT image. Furthermore, unless otherwise noted, the range gate marker is located at an initially set position (e.g., scan line position at the center of an ultrasonic image). The position of the range gate marker is changed depending on a process of the reception function 173, and this process is described later with reference to FIGS. 3A and 3B.

Furthermore, in accordance with the correspondence relation, the display control function 172 causes an angle correction marker for angle correction of blood-flow information to be displayed at a corresponding position on the display image based on the X-ray CT image data. For example, the display control function 172 causes the angle correction marker, which indicates the angle with respect to a scan line direction, to be displayed on an ultrasonic image and a 2D CT image. Furthermore, unless otherwise noted, the angle correction marker is located at an initially set angle (e.g., the right angle with respect to a scan line). The angle of the angle correction marker is changed depending on a process of the reception function 173, and this process is described later with reference to FIGS. 3A and 3B.

The reception function 173 receives, from the operator, an operation to set the range gate marker that indicates the position, from which blood-flow information is extracted, on the scan area of ultrasonic image data. Furthermore, the reception function 173 receives an angle change operation to change the angle of the angle correction marker on the display image. Here, the range gate marker is an example of a position marker. Furthermore, the angle correction marker is an example of an angle marker.

FIGS. 3A and 3B are diagrams that illustrate a process of the reception function 173 according to the first embodiment. FIG. 3A illustrates an example of the display screen before an operation is performed to set the range gate marker. Furthermore, FIG. 3B illustrates an example of the display screen after an operation is performed to set the range gate marker.

As illustrated in FIGS. 3A and 3B, the display control function 172 causes the display 103 to present an ultrasonic image 10, a 2D CT image 20, a Doppler waveform 30, and a measurement result 40. The display control function 172 causes a range gate marker 11 and an angle correction marker 12 to be displayed on the ultrasonic image 10. Furthermore, the display control function 172 causes a range gate marker 21, an angle correction marker 22, and a scan area marker 23 to be displayed on the 2D CT image 20. Here, the scan area marker 23 is a frame border that indicates the position of the ultrasonic image 10 on the 2D CT image 20. Furthermore, the Doppler waveform 30 is an example of the blood-flow information that is extracted from the sample volume, which is set at the position of the range gate marker 11. Furthermore, the measurement result 40 is a list of measurement values of measurement based on the waveform of the Doppler waveform 30.

Here, the display control function 172 locates the range gate marker 11 and the range gate marker 21 at a corresponding position (the same position) to each other. Specifically, after the range gate marker 11 is located on the ultrasonic image 10, the display control function 172 uses the correspondence relation, acquired by the acquisition function 171, to calculate the position that is on the 2D CT image 20 and that corresponds to the location position of the range gate marker 11. Then, the display control function 172 locates the range gate marker 21 at the calculated position. Furthermore, the display control function 172 locates the angle correction marker 12 and the angle correction marker 22 at the corresponding position and angle to each other. Specifically, after the angle correction marker 12 is located on the ultrasonic image 10, the display control function 172 uses the positional relationship, acquired by the acquisition function 171, to calculate the position that is on the 2D CT image 20 and that corresponds to the location position of the angle correction marker 12. Then, the display control function 172 locates the angle correction marker 22 at the calculated position. Furthermore, the display control function 172 locates the angle correction marker 22 at the same angle as the angle correction marker 12.

Here, the reception function 173 receives operations for setting the range gate markers 11, 21. For example, the positions of the range gate markers 11, 21 are related to the rotational position of the wheel that is provided on the operation panel. In this case, if the operator rotates the wheel to the left, the reception function 173 receives it as an operation to move the positions of the range gate markers 11, 21 to the left. Then, as illustrated in FIG. 3B, the display control function 172 moves the positions of the range gate markers 11, 21 to the left in accordance with an operation that is received by the reception function 173. Conversely, when the operator rotates the wheel to the right, the reception function 173 receives it as an operation to move the positions of the range gate markers 11, 21 to the right. Then, the display control function 172 moves the positions of the range gate markers 11, 21 to the right in accordance with the operation that is received by the reception function 173. In this way, the display control function 172 moves the positions of the two range gate markers 11, 21 in conjunction in accordance with a predetermined operation of the input device 102.

Furthermore, the reception function 173 receives operations (angle change operations) to change the angles of the angle correction markers 12, 22. For example, the angles of the angle correction markers 12, 22 are related to the rotation of the dial that is provided on the operation panel. In this case, when the operator rotates the dial to the right, the reception function 173 receives it as an operation to rotate the angles of the angle correction markers 12, 22 to the right. Then, the display control function 172 rotates the angles of the angle correction markers 12, 22 to the right in accordance with the operation that is received by the reception function 173. Conversely, when the operator rotates the dial to the left, the reception function 173 receives it as an operation to rotate the angles of the angle correction markers 12, 22 to the left. Then, the display control function 172 rotates the angles of the angle correction markers 12, 22 to the left in accordance with the operation that is received by the reception function 173. In this way, the display control function 172 rotates the angles of the two angle correction markers 12, 22 in conjunction in accordance with a predetermined operation of the input device 102.

In this way, the reception function 173 adjusts the range gate markers 11, 21 and the angle correction markers 12, 22. Furthermore, after the range gate markers 11, 21 are adjusted, the Doppler waveform 30 is collected at the adjusted position. Furthermore, after the angle correction markers 12, 22 are adjusted, the measurement result 40 is recalculated.

Here, the contents illustrated in FIGS. 3A and 3B are only an example, and the illustrated example is not a limitation. For example, with regard to the reception function 173, the input device 102, which receives operation from operators, are not limited to a wheel or a dial, and the input device 102 of any kind is applicable.

The calculation function 174 calculates a measurement value from blood-flow information. For example, the calculation function 174 calculates the velocity peak (VP) and the velocity time integral (VTI) by using an auto-trace function (or a manual-trace function) for Doppler waveforms. The measurement value calculated by the calculation function 174 is presented as the measurement result 40 on the display 103 by the display control function 172.

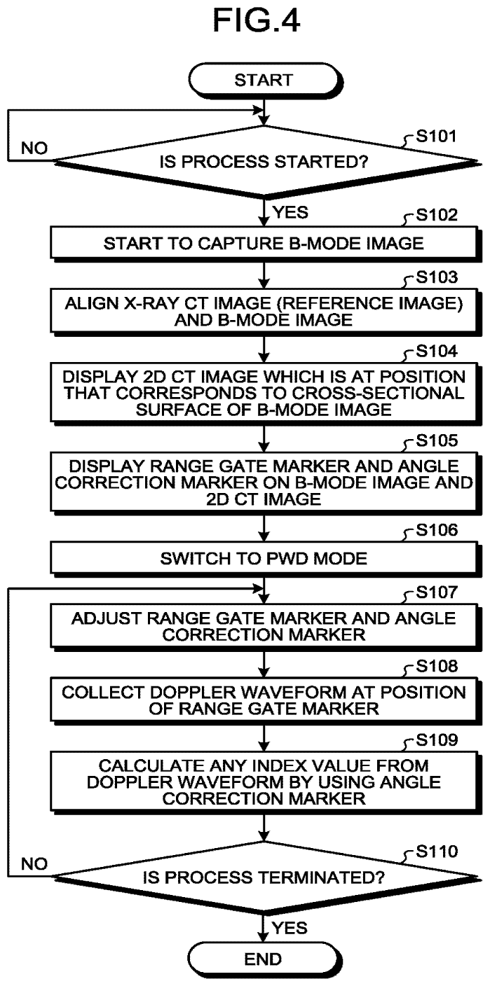

FIG. 4 is a flowchart that illustrates the steps of the process of the ultrasonic diagnostic device 1 according to the first embodiment. The procedure illustrated in FIG. 4 is started when, for example, a command is received to start a simultaneous display function so as to simultaneously display previously captured X-ray CT image data and ultrasonic image data.

As Step S101, the processing circuitry 170 determines whether the process is to be started. For example, the processing circuitry 170 determines that the process is to be started when a command to start a simultaneous display function is received from an operator (Yes at Step S101), and the process after Step S102 is started. Furthermore, if the process is not started (No at Step S101), the process after Step S102 is not started, and each processing function of the processing circuitry 170 is in a standby state.

If it is Yes at Step S101, the processing circuitry 170 starts to capture a B-mode image at Step S102. For example, the operator brings the ultrasonic probe 101 into contact with the body surface of the subject P and conducts ultrasonic scanning on the inside of the body of the subject P. The processing circuitry 170 controls the transmission/reception circuitry 110, the B-mode processing circuitry 120, the Doppler processing circuitry 130, and the image generation circuit 140 to capture ultrasonic images substantially in real time.

At Step S103, the acquisition function 171 aligns an X-ray CT image and a B-mode image. For example, the acquisition function 171 generates, as the positional relationship, the conversion function of the positional information on the B-mode image data in a three-dimensional space and the coordinate information on the X-ray CT image data. Furthermore, the X-ray CT image is previously read as a reference image and is presented on the display 103.

At Step S104, the display control function 172 causes the 2D CT image, which is at the position that corresponds to the cross-sectional surface of the B-mode image, to be displayed. For example, the display control function 172 uses the conversion function, generated by the acquisition function 171, to determine the cross-sectional position that is in the X-ray CT image data and that corresponds to the cross-sectional surface of the B-mode image. Then, the display control function 172 generates the 2D CT image, which corresponds to the determined cross-sectional position, through MPR processing, and presents it on the display 103.

At Step S105, the display control function 172 causes the range gate marker and the angle correction marker to be displayed on the B-mode image and the 2D CT image. For example, the display control function 172 causes the range gate marker and the angle correction marker to be displayed at corresponding positions on the B-mode image and the 2D CT image.

At Step S106, the processing circuitry 170 switches the capturing mode to the PWD mode. For example, the operator performs an operation to switch the capturing mode to the PWD mode so that the processing circuitry 170 starts to collect the blood-flow information in the PWD mode.

At Step S107, the reception function 173 adjusts the range gate marker and the angle correction marker. For example, when the wheel provided on the operation panel is rotated by the operator in a predetermined direction, the reception function 173 moves the range gate marker in a predetermined direction. Furthermore, when the dial provided on the operation panel is rotated by the operator in a predetermined direction, the reception function 173 rotates the angle correction marker with a predetermined angle.

At Step S108, the transmission/reception circuitry 110 and the Doppler processing circuitry 130 collect a Doppler waveform at the position of the range gate marker. For example, each time the position of the range gate marker is adjusted (changed), the processing circuitry 170 notifies the adjusted position to the transmission/reception circuitry 110 and the Doppler processing circuitry 130. Then, the transmission/reception circuitry 110 and the Doppler processing circuitry 130 transmit and receive ultrasonic pulses with respect to the notified position and extract a Doppler waveform from the received reflected-wave data. The extracted Doppler waveform is presented on the display 103 by the display control function 172.

At Step S109, the calculation function 174 calculates any index value (measurement value) from the Doppler waveform by using the angle correction marker. For example, each time the angle of the angle correction marker is changed, the calculation function 174 corrects the Doppler waveform by using the angle of the angle correction marker (the angle of the angle correction marker with respect to a scan line). Then, the calculation function 174 recalculates the measurement value, which is the measurement target, on the basis of the corrected Doppler waveform. The recalculated measurement value is presented on the display 103 by the display control function 172.

At Step S110, the processing circuitry 170 determines whether the process is terminated. For example, the processing circuitry 170 determines that the process is terminated if a command to terminate the simultaneous display function is received from the operator (Yes at Step S110) and terminates the procedure of FIG. 4. Furthermore, if the process is not terminated (No at Step S110), the processing circuitry 170 proceeds to the operation at Step S107. That is, the processing circuitry 170 may receive adjustments of the range gate marker and the angle correction marker until the process is terminated.

Here, the contents illustrated in FIG. 4 are only an example, and this is not a limitation on the embodiment. In the illustrated case according to the above-described procedure, the range gate marker is adjusted after collection of blood-flow information in the PWD mode is started; however, this is not a limitation on the embodiment. For example, collection of blood-flow information in the PWD mode may be started after the position of the range gate marker is adjusted to an appropriate position.

As described above, the ultrasonic diagnostic device 1 according to the first embodiment includes the ultrasonic probe 101, the acquisition function 171, the reception function 173, and the display control function 172. The ultrasonic probe 101 conducts ultrasonic scanning on the subject P to receive reflected waves from the subject P. The acquisition function 171 acquires the correspondence relation between a position in the ultrasonic image data based on the reflected waves and a position in the volume data on the subject P, captured by a different medical-image diagnostic device. The reception function 173 receives, from the operator, an operation to set the position marker that indicates the position, from which blood-flow information is extracted, on the scan area of the ultrasonic image data. On the basis of the correspondence relation, the display control function 172 causes the position marker to be displayed at a corresponding position on the display image based on at least the volume data. Thus, the ultrasonic diagnostic device 1 according to the first embodiment may improve for example the accuracy and the quantitative characteristic of blood-flow information.

For example, the ultrasonic diagnostic device 1 according to the first embodiment may adjust the positions of the two range gate markers, displayed on the ultrasonic image and the 2D CT image, in conjunction with each other. Thus, for example, the operator may adjust the position of the range gate marker by operating the input device 102 while checking the position of the range gate marker on the 2D CT image. Generally, it is considered that 2D CT images have superior accuracy as form information. Therefore, operators may adjust the position of the range gate marker with more accuracy and collect blood-flow information at a desired position with accuracy.

Furthermore, for example, the ultrasonic diagnostic device 1 according to the first embodiment may adjust the angles of the two angle correction markers, displayed on the ultrasonic image and the 2D CT image, in conjunction with each other. Thus, for example, the operator may adjust the angle of the angle correction marker by operating the input device 102 while checking the angle of the angle correction marker on the 2D CT image. Hence, operators may properly adjust the angle of the angle correction marker and may obtain blood-flow information with improved quantitative characteristic.

Thus, the ultrasonic diagnostic device 1 may provide blood-flow information with superior accuracy and quantitative characteristic for cases, such as mitral valve regurgitation, atrial septal defect, aortic valve regurgitation, coronary artery embolism, or truncus arteriosus communis.

Furthermore, the contents described in the first embodiment are only an example, and the above-described contents are not always a limitation. With reference to the drawings, an explanation is given below of a modified example of the first embodiment.

### Modified Example 1 of the First Embodiment

In the first embodiment, an explanation is given of a case where the range gate marker and the angle correction marker are adjusted in accordance with an operation of the input device 102; however, this is not a limitation on the embodiment. For example, according to the embodiment, there may be a case where a UI is provided to change the range gate marker and the angle correction marker on the display image of X-ray CT image data and adjustments are made by using the UI.

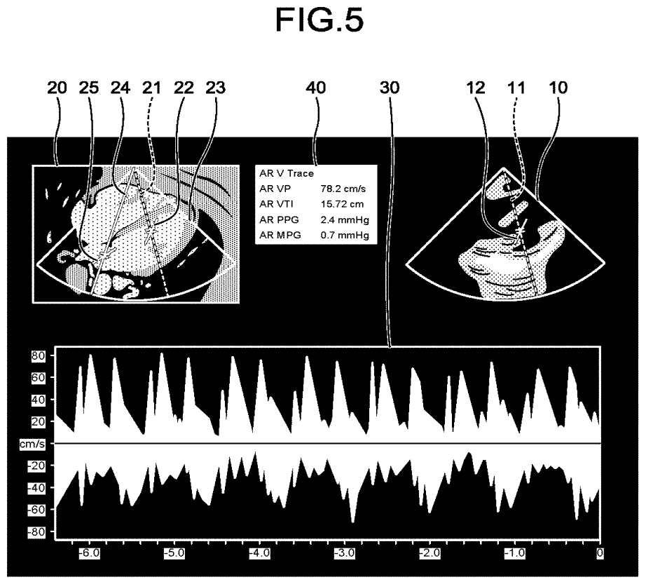

FIG. 5 is a diagram that illustrates a process of the reception function 173 according to the modified example 1 of the first embodiment. FIG. 5 illustrates a case where the UI is used to adjust the range gate marker and the angle correction marker on a 2D CT image. Furthermore, as the ultrasonic image 10, the Doppler waveform 30, and the measurement result 40 illustrated in FIG. 5 are the same as those in FIG. 3A, their explanations are omitted.

As illustrated in FIG. 5, the display control function 172 causes the range gate marker 21, the angle correction marker 22, the scan area marker 23, a position adjustment marker 24, and an angle adjustment marker 25 to be displayed on the 2D CT image 20. Here, as the range gate marker 21, the angle correction marker 22, and the scan area marker 23 are the same as those in FIG. 3A, their explanations are omitted.

Here, the position adjustment marker 24 is a marker used to adjust the positions of the range gate markers 11, 21. Furthermore, the angle adjustment marker 25 is a marker used to adjust the angles of the angle correction markers 12, 22.

For example, if the operator inputs a command to adjust the positions of the range gate markers 11, 21 or the angles of the angle correction markers 12, 22, the reception function 173 causes the position adjustment marker 24 and the angle adjustment marker 25 to be displayed on the 2D CT image 20. Then, the operator operates the input device 102 (wheel, dial, mouse, keyboard, or the like) of any kind to change the position of the position adjustment marker 24 or the angle of the angle adjustment marker 25. At this stage, the positions of the range gate markers 11, 21 and the angles of the angle correction markers 12, 22 are not changed, and only the position of the position adjustment marker 24 and the angle of the angle adjustment marker 25 are changed on the 2D CT image 20. If it is determined that the position adjustment marker 24 is set at an appropriate position as the position of the range gate marker and if it is determined that the angle adjustment marker 25 is set at an appropriate angle as the angle of the angle correction marker, the operator presses the confirmation button. Thus, the reception function 173 moves the range gate markers 11, 21 to the position of the position adjustment marker 24 and rotates the angle correction markers 12, 22 to the angle of the angle adjustment marker 25.

In this manner, the reception function 173 receives an operation to set the position of the range gate marker on the display image of X-ray CT image data. Furthermore, the reception function 173 receives an operation to set the angle of the angle correction marker on the display image of X-ray CT image data. Therefore, operators may change, for example, the range gate marker and the angle correction marker on the display image of X-ray CT image data. Thus, operators may adjust the range gate marker and the angle correction marker on a 2D CT image, which has superior accuracy as form information, and therefore may collect blood-flow information at a desired position with accuracy.

Here, the contents illustrated in FIG. 5 are only an example, and the illustrated contents are not a limitation. For example, although FIG. 5 illustrates a case where both the position adjustment marker 24 and the angle adjustment marker 25 are simultaneously confirmed, this is not a limitation and, for example, there may be a case where the position adjustment marker 24 and the angle adjustment marker 25 are individually confirmed (the confirmation button is press).

### A Modified Example 2 of the First Embodiment

Furthermore, for example, each time the angle of the angle correction marker is changed, the display control function 172 may display the measurement value of blood-flow information, whose angle has been corrected at the changed angle, on a different display area.

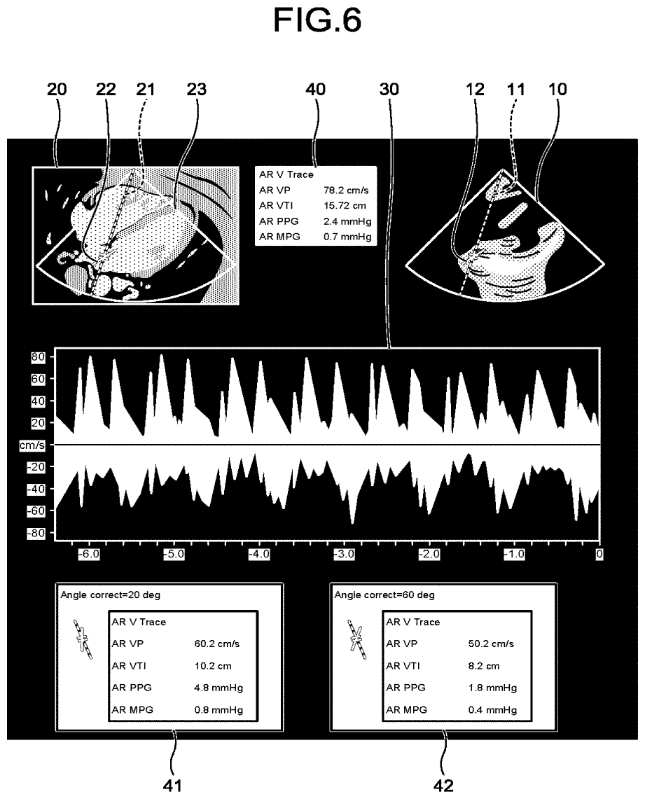

FIG. 6 is a diagram that illustrates a process of the display control function 172 according to the modified example 2 of the first embodiment. FIG. 6 illustrates an example of the display screen presented on the display 103 due to the process of the display control function 172. Here, as the ultrasonic image 10, the 2D CT image 20, the Doppler waveform 30, and the measurement result 40 in FIG. 6 are the same as those in FIG. 3B, their explanations are omitted.

For example, in some cases, it is difficult for an operator to determine the angles of the angle correction markers 12, 22, at which an accurate measurement value is obtained. In such a case, the operator performs an operation to hold a measurement result at the angle that is supposed to be accurate. For example, if it is determined that an accurate measurement value is obtained when the angles of the angle correction markers 12, 22 are 20 degrees, the operator presses the hold button (the first press). Thus, the display control function 172 displays a measurement result 41 on the display 103. The measurement result 41 includes a measurement value when the angles of the angle correction markers 12, 22 are 20 degrees and the icon of the angle correction markers 12, 22.

Furthermore, for example, if it is determined that an accurate measurement value is obtained when the angles of the angle correction markers 12, 22 are 60 degrees, the operator presses the hold button (the second press). Thus, the display control function 172 presents a measurement result 42 on the display 103. The measurement result 42 includes a measurement value when the angles of the angle correction markers 12, 22 are 60 degrees and the icon of the angle correction markers 12, 22.

In this way, each time the angle of the angle correction marker is changed, the calculation function 174 presents the measurement value of blood-flow information, whose angle has been corrected at the changed angle, on a different display area. Thus, operators may subsequently determine whether an accurate measurement value is obtained.

Here, the contents illustrated in FIG. 6 are only examples, and the illustrated contents are not a limitation. For example, FIG. 6 illustrates a case where two measurement results are held; however, this is not a limitation, and the number of measurement results to be held may be optionally set.

### A Modified Example 3 of the First Embodiment

Furthermore, for example, the calculation function 174 may use a first measurement value, measured from ultrasonic image data or blood-flow information, and a second measurement value, measured from volume data, to calculate the index value related to the subject P.

For example, the calculation function 174 uses the following Equation (1) to calculate left ventricular outflow tract stroke volume LVOT SV [mL]. Here, in Equation (1), LVOT Diam denotes the left ventricular outflow tract diameter. Furthermore, LVOT VTI denotes the time velocity integral of blood flow waveform in the left ventricular outflow tract.

$$LVOT SV = \frac{π}{4} {( LVOT Diam )}^{2} \times \frac{\vert LVOT VTI \vert}{100} \qquad ( 1 )$$

Here, the calculation function 174 uses the left ventricular outflow tract diameter, calculated from the 2D CT image 20, as LVOT Diam in Equation (1). Furthermore, the calculation function 174 uses the time velocity integral of the blood flow waveform in the left ventricular outflow tract, calculated from blood-flow information, as LVOT VTI in Equation (1).

In this way, the calculation function 174 applies LVOT VTI, measured from blood-flow information, and LVOT Diam, measured from the 2D CT image 20, to Equation (1) to calculate the left ventricular outflow tract stroke volume LVOT SV. For example, if LVOT Diam is measured from an ultrasonic image, a circular cross-sectional surface is estimated and calculated. Conversely, if LVOT Diam is measured from a 2D CT image, a cross-sectional area in the image may be calculated with accuracy. Therefore, the calculation function 174 may calculate the left ventricular outflow tract stroke volume LVOT SV with more accuracy.

Furthermore, the calculation function 174 may calculate not only the left ventricular outflow tract stroke volume LVOT SV but also other index values. For example, the calculation function 174 uses the following Equation (2) to calculate mitral valve stroke volume MV SV [mL]. Here, in Equation (2), MV DistA denotes mitral valve diameter A. MV DistB denotes mitral valve diameter B. Furthermore, MV VTI denotes the time velocity integral of a blood flow waveform in the mitral valve.

$$MVSV = \frac{π}{4} \times \frac{( MV DistA )}{10} \times \frac{( MV DistB )}{10} \times \vert MV VTI \vert \qquad ( 2 )$$

Here, the calculation function 174 uses the mitral valve diameter A and the mitral valve diameter B, calculated from the 2D CT image 20, as MV DistA and MV DistB in Equation (2). Furthermore, the calculation function 174 uses the time velocity integral of the blood flow waveform in the mitral valve, calculated from blood-flow information, as MV VTI in Equation (2).

In this way, the calculation function 174 applies MV VTI, measured from the blood-flow information, and MV DistA and MV DistB, measured from the 2D CT image 20, to Equation (2), thereby calculating the mitral valve stroke volume MV SV.

Furthermore, in the modified example 3 of the first embodiment, an explanation is given of a case where the stroke volume is measured as the index value related to the subject P; however, this is not a limitation on the embodiment.

### Second Embodiment

In the first embodiment, an explanation is given of a case where the 2D CT image, which is two-dimensional X-ray CT image data, is displayed; however, this is not a limitation on the embodiment. For example, the ultrasonic diagnostic device 1 may display other rendering images, which are generated from volume data, which is three-dimensional X-ray CT image data, during a rendering process.

The ultrasonic diagnostic device 1 according to the second embodiment has the same configuration as the ultrasonic diagnostic device 1 illustrated in FIG. 1, and part of the process of the display control function 172 is different. Therefore, the second embodiment is primarily explained in the part that is different from the first embodiment, and explanations are omitted for the part that has the same function as the configuration explained in the first embodiment.

The display control function 172 according to the second embodiment causes a rendering image, which is generated during a rendering process on volume data, which is three-dimensional X-ray CT image data, to be displayed. Furthermore, the display control function 172 causes the cross-sectional position that corresponds to the B-mode image and the cross-sectional position that corresponds to the 2D CT image to be displayed on the rendering image. Furthermore, the display control function 172 causes the range gate marker and the angle correction marker to be displayed on the rendering image.

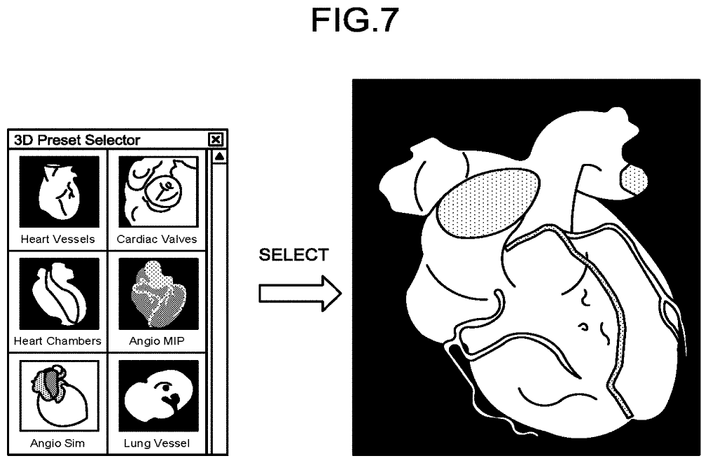

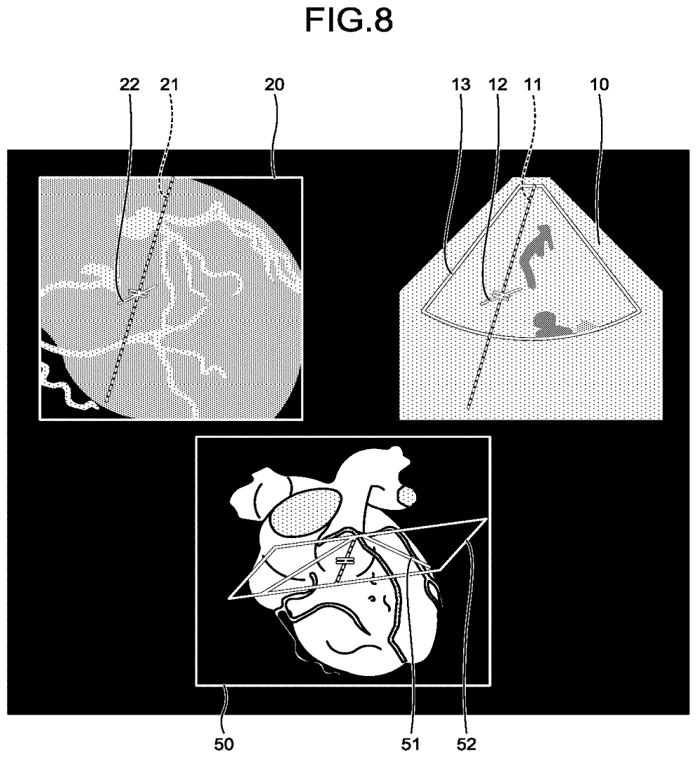

FIGS. 7 and 8 are diagrams that illustrate the process of the display control function 172 according to the second embodiment. FIG. 7 illustrates an example of the process to generate segmentation data, previously performed on volume data. Furthermore, FIG. 8 illustrates an example of the display screen that is presented on the display 103.

As illustrated in FIG. 7, segmentation is previously conducted on the volume data, stored in the image memory 150, and it is generated as an image where various types of tissues are color-coded in accordance with a diagnosis purpose. For example, as illustrated in the left section of FIG. 7, the operator selects a display mode, in which a desired tissue is displayed, from multiple choices. Thus, as illustrated in the right section of FIG. 7, the volume data is generated as the volume rendering image (or surface rendering image) where, for example, the tissues including the heart and the coronary artery are color-coded.

As illustrated in FIG. 8, the display control function 172 causes the ultrasonic image 10, the 2D CT image 20, and a volume rendering image 50 to be presented on the display 103. Here, the display control function 172 causes the range gate marker 11, the angle correction marker 12, and a color region of interest (ROI) 13 to be presented on the ultrasonic image 10. The color ROI 13 is an area where a blood flow image is presented by being rendered according to a color Doppler technique, and the coronary artery blood flow is displayed in the example of FIG. 8. That is, the ultrasonic probe 101 conducts ultrasonic scanning on the area that includes the coronary artery of the subject P. Then, the display control function 172 causes the ultrasonic image, where the coronary artery is rendered, to be displayed.

Furthermore, the display control function 172 causes the range gate marker 21 and the angle correction marker 22 to be displayed on the 2D CT image 20. Here, the 2D CT image 20 is a cross-sectional image that is in the volume data and that is at the position that corresponds to the ultrasonic image 10.

Here, the display control function 172 causes a scan area marker 51 and a cross-section position marker 52 to be displayed on the volume rendering image 50. The scan area marker 51 is a frame border that is on the volume rendering image 50 and that indicates the position of the ultrasonic image 10. Furthermore, the cross-section position marker 52 is a frame border that is on the volume rendering image 50 and that indicates the position of the 2D CT image 20. Furthermore, as illustrated in FIG. 8, the display control function 172 may cause the marker that corresponds to the range gate marker 11 or the marker that corresponds to the angle correction marker 12 to be displayed on the volume rendering image 50.

In this way, the ultrasonic diagnostic device 1 according to the second embodiment may cause a volume rendering image, generated from volume data that is three-dimensional X-ray CT image data, to be displayed and further cause the range gate marker, the angle correction marker, the scan area marker, and the cross-section position marker to be displayed on a volume rendering image. This allows operators to known the position of the range gate marker, the angle of the angle correction marker, the position of the scan area, and the position of the 2D CT image on the image presented in three dimensions.

Here, the contents illustrated in FIG. 8 are only examples, and the illustrated contents are not a limitation. For example, in FIG. 8, an explanation is given of a case where the volume rendering image 50, on which the entire heart is rendered, is displayed as a rendering image; however, this is not a limitation and, for example, it is possible to display a volume rendering image where only the coronary artery is rendered. Furthermore, in addition to the image illustrated in FIG. 8, the display control function 172 may cause the Doppler waveform 30 and the measurement result 40 to be displayed.

Here, the contents explained in the second embodiment are the same as those explained in the first embodiment except that the display control function 172 causes rendering images to be displayed other than cross-sectional images. That is, the configuration and the modified examples described in the first embodiment are applicable to the second embodiment except that the display control function 172 displays rendering images other than cross-sectional images.

### Third Embodiment

In the above-described embodiment, although an explanation is given of a case where two-dimensional ultrasonic images are displayed, this is not a limitation on the embodiment. For example, if ultrasonic scanning is conducted on three-dimensional areas, the ultrasonic diagnostic device 1 may display rendering images of ultrasonic waves, generated during a rendering process on three-dimensional ultrasonic image data.

The ultrasonic diagnostic device 1 according to the third embodiment has the same configuration as the ultrasonic diagnostic device 1 illustrated in FIG. 1, and part of processes of the ultrasonic probe 101 and the display control function 172 is different. Therefore, the third embodiment is primarily explained in the part that is different from the above-described embodiments, and explanations are omitted for the part that has the same function as the configuration explained in the above-described embodiments.

The ultrasonic probe 101 according to the third embodiment conducts ultrasonic scanning on a three-dimensional area of the subject P. In this case, the transmission/reception circuitry 110 causes the ultrasonic probe 101 to transmit three-dimensional ultrasonic beams. Then, the transmission/reception circuitry 110 generates three-dimensional reflected-wave data from the three-dimensional reflected-wave signal that is received from the ultrasonic probe 101. Then, the B-mode processing circuitry 120 generates three-dimensional B mode data from the three-dimensional reflected-wave data. Furthermore, the Doppler processing circuitry 130 generates three-dimensional Doppler data from the three-dimensional reflected-wave data. Then, the image generation circuit 140 generates three-dimensional B-mode image data from the three-dimensional B mode data and generates three-dimensional Doppler image data from the three-dimensional Doppler data.

The display control function 172 according to the third embodiment causes rendering images of ultrasonic waves, generated during a rendering process on the ultrasonic image data on the three-dimensional area, to be displayed. For example, the display control function 172 causes volume rendering images or surface rendering images to be presented as rendering images of ultrasonic waves on the display 103.

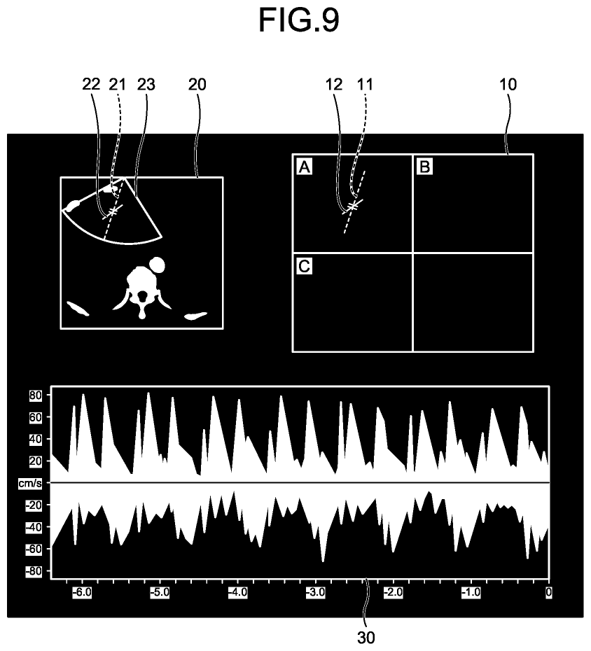

FIG. 9 is a diagram that illustrates a process of the display control function 172 according to the third embodiment. FIG. 9 illustrates an example of the display screen presented on the display 103. Furthermore, as the Doppler waveform 30 in FIG. 9 is the same as that in FIG. 3A, or the like, explanations are omitted.

As illustrated in FIG. 9, the display control function 172 causes the ultrasonic image 10 and the 2D CT image 20 to be presented on the display 103. For example, the display control function 172 causes the volume rendering image, which is a color Doppler image that captures the portal vein of the liver, and the cross-sectional images of side A, side B, and side C to be displayed as the ultrasonic image 10. Here, on the cross-sectional images of the side A, the side B, and the side C, B-mode images are rendered as background images. Furthermore, the display control function 172 causes the range gate marker 11 and the angle correction marker 12 to be displayed on the cross-sectional image of the side A.

Furthermore, the display control function 172 causes the range gate marker 21, the angle correction marker 22, and the scan area marker 23 to be displayed on the 2D CT image 20. Here, on the 2D CT image 20, the range gate marker 21 and the angle correction marker 22 are markers that correspond to the positions and the angles of the range gate marker 11 and the angle correction marker 12. Furthermore, the scan area marker 23 is a frame border that indicates the position of the cross-sectional image of the side A on the 2D CT image 20.

In this way, the ultrasonic diagnostic device 1 according to the third embodiment may further display rendering images of ultrasonic waves, generated during a rendering process on three-dimensional ultrasonic image data.

Furthermore, the contents illustrated in FIG. 9 are only examples, and the illustrated contents are not a limitation. For example, the display control function 172 may cause the range gate marker 11 and the angle correction marker 12 to be displayed on a volume rendering image (or surface rendering image). In this case, it is preferable that volume rendering images are volume rendering images (or surface rendering images) that represent living tissues that are cut on any cross-sectional surface and the range gate marker 11 and the angle correction marker 12 are displayed on the cross-sectional surface.

Here, the contents explained in the third embodiment are the same as those explained in the above-described embodiments except that the display control function 172 causes rendering images of ultrasonic waves to be displayed. That is, the configurations and the modified examples described in the above-described embodiments are applicable to the third embodiment except that the display control function 172 displays rendering images of ultrasonic waves.

### Fourth Embodiment

In the above-described embodiment, an explanation is given of a case where ultrasonic images are displayed substantially in real time; however, this is not a limitation on the embodiment. For example, if electrocardiographic signals of the subject P may be detected, the ultrasonic diagnostic device 1 may display ultrasonic images in the cardiac time phase that is substantially identical to the cardiac time phase of X-ray CT image data.

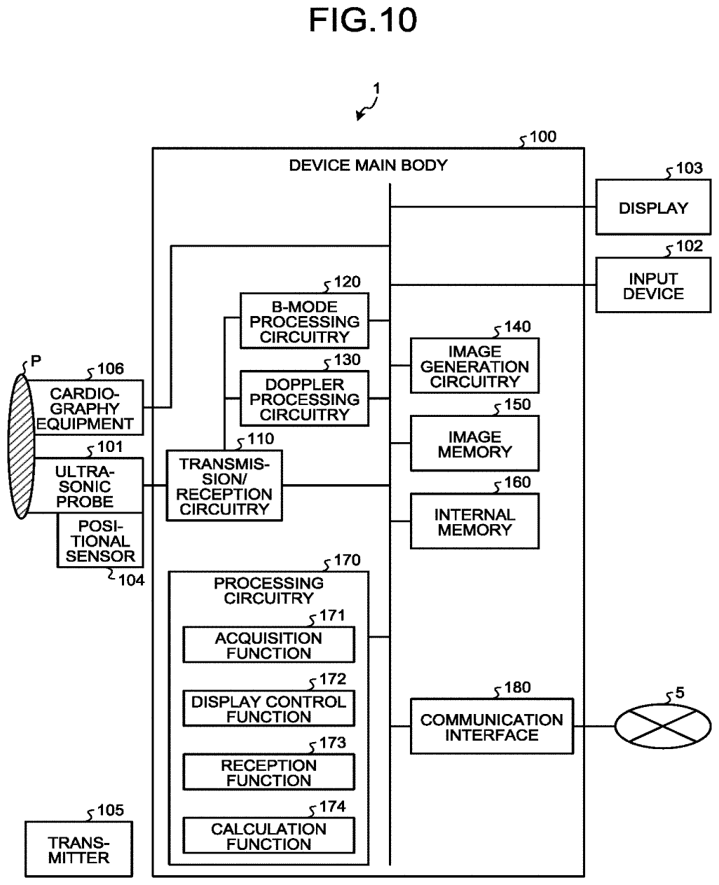

FIG. 10 is a block diagram that illustrates an example of the configuration of the ultrasonic diagnostic device 1 according to the fourth embodiment. As illustrated in FIG. 10, the ultrasonic diagnostic device 1 according to the fourth embodiment further includes cardiography equipment 106 in addition to the same configuration as that of the ultrasonic diagnostic device 1 illustrated in FIG. 1. The fourth embodiment is primarily explained in the part that is different from the above-described embodiments, and explanations are omitted for the part that has the same function as the configurations explained in the above-described embodiments.

The cardiography equipment 106 according to the fourth embodiment is equipment that detects electrocardiographic signals of the subject P. For example, the cardiography equipment 106 acquires electrocardiographic waveforms (electrocardiogram: ECG) of the subject P as biosignals of the subject P that undergoes ultrasonic scanning. The cardiography equipment 106 transmits acquired electrocardiographic waveforms to the device main body 100. Furthermore, the electrocardiographic signals detected by the cardiography equipment 106 are stored in the internal memory 160 in relation to the capturing time of ultrasonic image data (the time when ultrasonic scanning is conducted to generate the ultrasonic image data). Thus, each frame of captured ultrasonic image data is related to a cardiac time phase of the subject P.

Here, in the present embodiment, an explanation is given of a case where the cardiography equipment 106 is used as a unit that acquires the information about a cardiac time phase of the heart of the subject P; however, this is not a limitation on the embodiment. For example, the ultrasonic diagnostic device 1 may acquire the information about a cardiac time phase of the heart of the subject P by acquiring the time of the II sound (the second sound) of phonocardiogram or the aortic valve close (AVC) time that is obtained by measuring the ejected blood flow of the heart due to the spectrum Doppler. Furthermore, for example, the ultrasonic diagnostic device 1 may extract the timing when the heart valve opens and closes during image processing on the captured ultrasonic image data and acquire a cardiac time phase of the subject in accordance with the timing. In other words, the processing circuitry 170 of the ultrasonic diagnostic device 1 may perform a cardiac time-phase acquisition function to acquire a cardiac time phase of the subject. Here, the cardiac time-phase acquisition function is an example of a cardiac time-phase acquiring unit. Furthermore, the cardiography equipment 106 is an example of a detecting unit.

On the basis of electrocardiographic signals, the display control function 172 according to the fourth embodiment displays ultrasonic images in the cardiac time phase that is substantially identical to the cardiac time phase of the medical image data captured by a different medical-image diagnostic device. For example, the display control function 172 displays B-mode images, generated substantially in real time, and also displays B-mode images in the cardiac time phase that is substantially identical to the cardiac time phase (e.g., end diastole) of X-ray CT image data.

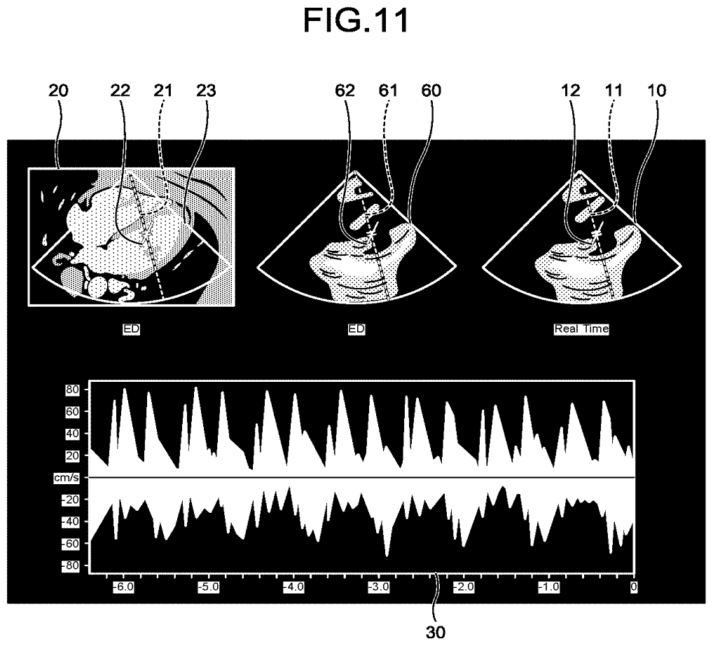

FIG. 11 is a diagram that illustrates a process of the display control function 172 according to the fourth embodiment. FIG. 11 illustrates an example of the display screen presented on the display 103 due to the process of the display control function 172. Here, FIG. 11 illustrates a case where a cardiac time phase of X-ray CT image data is end diastole (ED).

As illustrated in FIG. 11, the display control function 172 causes the ultrasonic image 10, the 2D CT image 20, and the Doppler waveform 30 to be displayed. Here, the ultrasonic image 10 is an image substantially in real time, and the 2D CT image 20 is an image at the end diastole (ED). Here, as the details of the ultrasonic image 10, the 2D CT image 20, and the Doppler waveform 30 are the same as those in FIG. 3A, explanations are omitted.

Here, if the cardiac time phase of X-ray CT image data is the end diastole (ED), the display control function 172 causes an ultrasonic image 60, whose cardiac time phase is the end diastole (ED), to be displayed in accordance with electrocardiographic signals. For example, the display control function 172 refers to the electrocardiographic signal (electrocardiographic waveform), detected by the cardiography equipment 106, and determines the time that corresponds to the end diastole. Then, the display control function 172 uses the ultrasonic image data, which corresponds to the determined time, to generate the ultrasonic image 60 for display and causes it to be presented on the display 103. Afterward, each time an electrocardiographic signal that indicates the end diastole is detected, the display control function 172 generates the ultrasonic image 60 that corresponds to the detected time and updates the ultrasonic image 60 presented on the display 103.

Furthermore, the display control function 172 causes a range gate marker 61 and an angle correction marker 62 to be displayed on the ultrasonic image 60 at the end diastole (ED). Specifically, the display control function 172 causes the range gate marker 61 to be displayed at the position that corresponds to the range gate markers 11, 21 and causes the angle correction marker 62 to be displayed at the angle that corresponds to the angle correction markers 12, 22.

In this way, the display control function 172 causes an ultrasonic image to be displayed in the cardiac time phase that is substantially identical to the cardiac time phase of different medical image data, displayed with a simultaneous display function. Thus, for example, an operator may adjust the range gate marker and the angle correction marker while simultaneously referring to a 2D CT image and an ultrasonic image, whose cardiac time phases are matched.

Here, the contents illustrated in FIG. 11 are only an example, and the illustrated contents are not a limitation. For example, the display control function 172 does not always need to display the ultrasonic image 10 substantially in real time. Even if the ultrasonic image 10 is not displayed substantially in real time, the operator may adjust the range gate marker and the angle correction marker while simultaneously referring to a 2D CT image and an ultrasonic image, whose cardiac time phases are matched. Furthermore, instead of the ultrasonic image 60 at the end diastole (ED), the display control function 172 may display ultrasonic images at the end systole (ES) and may simultaneously display ultrasonic images at three or more different time phases on the display 103.

Here, the contents explained in the fourth embodiment are the same as those explained in the above-described embodiments except that the display control function 172 displays an ultrasonic image in the cardiac time phase that is substantially identical to the cardiac time phase of X-ray CT image data. That is, the configuration and the modified examples described in the above-described embodiments are applicable to the fourth embodiment except that the display control function 172 displays an ultrasonic image in the cardiac time phase that is substantially identical to the cardiac time phase of X-ray CT image data.

### Fifth Embodiment

In the above-described embodiment, an explanation is given of a case where the range gate marker and the angle correction marker are adjusted on a cross-sectional image (ultrasonic image or 2D CT image); however, this is not a limitation on the embodiment. For example, the ultrasonic diagnostic device 1 may receive an operation to adjust a range gate marker on a rendering image that is displayed in three dimensions.

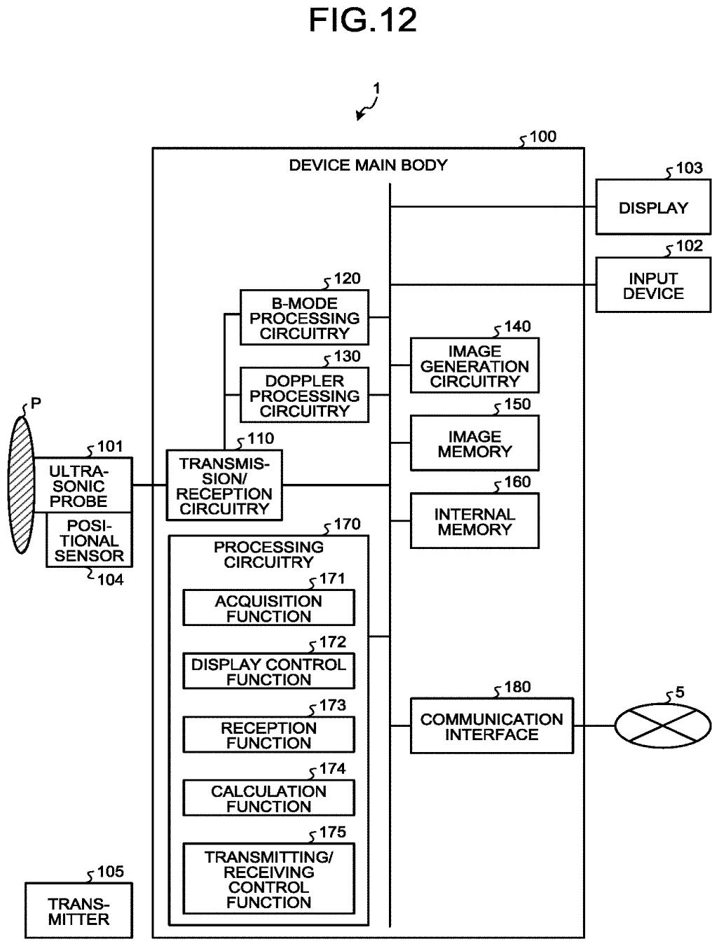

FIG. 12 is a block diagram that illustrates an example of the configuration of the ultrasonic diagnostic device 1 according to the fifth embodiment. As illustrated in FIG. 12, the ultrasonic diagnostic device 1 according to the fifth embodiment further includes a transmitting/receiving control function 175 in the processing circuitry 170 in addition to the same configuration as that of the ultrasonic diagnostic device 1 illustrated in FIG. 1. Therefore, the fifth embodiment is primarily explained in the part that is different from the above-described embodiments, and explanations are omitted for the part that has the same function as the configuration explained in the above-described embodiments.

The ultrasonic probe 101 according to the fifth embodiment is a two-dimensional array probe. For example, if scanning is conducted on a two-dimensional scan cross-sectional surface, the ultrasonic probe 101 may change the direction of the scan cross-sectional surface with respect to the ultrasonic probe 101. That is, the operator may change (deflect) the direction of a scan cross-sectional surface without changing the position or the direction of the ultrasonic probe 101 that is in contact with the body surface of the subject P.

The transmitting/receiving control function 175 according to the fifth embodiment performs a control to change the direction of the scan cross-sectional surface, on which the ultrasonic probe 101 conducts scanning. For example, if the operator gives a command to tilt the scan cross-sectional surface at 5 degrees in the elevation angle direction, the transmitting/receiving control function 175 transmits the command to tilt the scan cross-sectional surface at 5 degrees in the elevation angle direction to the ultrasonic probe 101. Thus, the ultrasonic probe 101 tilts the scan cross-sectional surface at 5 degrees in the elevation angle direction.

The display control function 172 according to the fifth embodiment displays rendering images generated during a rendering process on volume data, which is three-dimensional X-ray CT image data. Here, as the display control function 172 according to the fifth embodiment performs the same process as that of the display control function 172 according to the second embodiment, explanations are omitted.

The reception function 173 according to the fifth embodiment receives an operation to change the position of the position marker on a rendering image. For example, the reception function 173 receives a setting operation to set the range gate marker on the rendering image generated by the display control function 172.

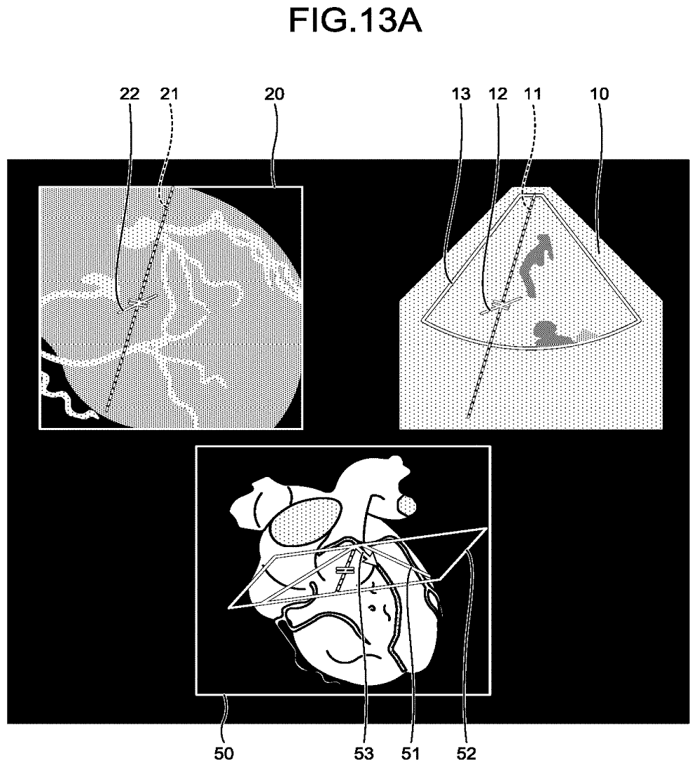

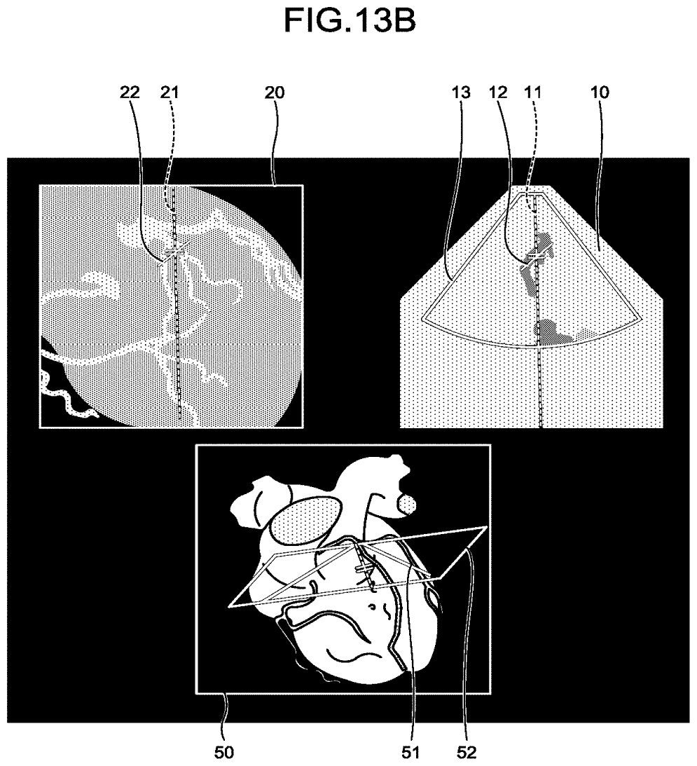

FIGS. 13A and 13B are diagrams that illustrate a process of the reception function 173 according to the fifth embodiment. FIG. 13A illustrates an example of the display screen before an operator performs a setting operation. Furthermore, FIG. 13B illustrates an example of the display screen after an operator performs a setting operation.

As illustrated in FIG. 13A, the display control function 172 causes the ultrasonic image 10, the 2D CT image 20, and the volume rendering image 50 to be displayed. Here, as the details of the ultrasonic image 10 and the 2D CT image 20 are the same as those in FIG. 8, their explanations are omitted.

Here, the display control function 172 causes a position-adjustment marker 53 to be displayed as an UI for adjusting the range gate marker on the volume rendering image 50.

For example, if the operator inputs a command to adjust the positions of the range gate markers 11, 21, the reception function 173 causes the position-adjustment marker 53 to be displayed on the volume rendering image 50. Then, the operator operates the input device 102 (wheel, dial, mouse, keyboard, or the like) of any kind to change the position of the position-adjustment marker 53. For example, any coordinates are designated on the volume rendering image 50 by using the mouse cursor so that the coordinates on the end of the position-adjustment marker 53 are designated. At this stage, the positions of the range gate markers 11, 21 are not changed, and only the position of the position-adjustment marker 53 is changed on the volume rendering image 50. If it is determined that the position-adjustment marker 53 is set at an appropriate position as the positions of the range gate markers 11, 21, the operator presses the confirmation button. After the confirmation button is pressed, the reception function 173 receives it as an operation to set the range gate markers 11, 21 at the coordinates (hereafter, also referred to as the “designated coordinates”) designated by the operator.

Then, the reception function 173 determines whether the designated coordinates are present on the scan cross-sectional surface (on the ultrasonic image 10). If the designated coordinates are not present on the scan cross-sectional surface, the reception function 173 notifies the designated coordinates to the transmitting/receiving control function 175.

After the designated coordinates are notified by the reception function 173, the transmitting/receiving control function 175 changes the direction of the scan cross-sectional surface such that the notified designated coordinates are included in the scan cross-sectional surface. For example, the transmitting/receiving control function 175 calculates the angle (the elevation angle or the depression angle) of the scan cross-sectional surface that passes the designated coordinates. Then, the transmitting/receiving control function 175 performs control to tilt the scan cross-sectional surface until the calculated angle. In this manner, the ultrasonic probe 101 tilts the scan cross-sectional surface such that the scan cross-sectional surface passes the designated coordinates. Then, as illustrated in FIG. 13B, the reception function 173 moves the range gate markers 11, 21 to the position that passes the designated coordinates on the tilted scan cross-sectional surface (the ultrasonic image 10).

Conversely, if the designated coordinates are present on the scan cross-sectional surface (on the ultrasonic image 10), the reception function 173 moves the range gate markers 11, 21 to the position that passes the designated coordinates on the scan cross-sectional surface. In this case, the transmitting/receiving control function 175 does not perform control to change the direction of the scan cross-sectional surface.

In this way, the reception function 173 receives an operation to change the positions of the range gate markers 11, 21 on the volume rendering image 50. Then, the transmitting/receiving control function 175 performs control to change the direction of the scan cross-sectional surface such that the positions of the range gate markers 11, 21, which have been changed due to an operation, are included on the scan cross-sectional surface. Then, the reception function 173 moves the range gate markers 11, 21 to the position that passes the designated coordinates on the scan cross-sectional surface whose direction has been changed. This allows an operator to adjust the range gate marker on the volume rendering image 50, which has superior accuracy as form information, whereby blood-flow information at a desired position may be collected accurately and easily.

Here, the contents illustrated in FIGS. 13A and 13B are only an example, and the illustrated contents are not a limitation. For example, in FIGS. 13A and 13B, an explanation is given of a case where the volume rendering image 50, on which the entire heart is rendered, is displayed as a rendering image; however, this is not a limitation and, for example, it is possible to display a volume rendering image where only the coronary artery is rendered. Furthermore, in addition to the image illustrated in FIGS. 13A and 13B, the display control function 172 may cause the Doppler waveform 30 and the measurement result 40 to be displayed.

Here, the contents explained in the fifth embodiment are the same as those explained in the above-described embodiments except that the reception function 173 receives an operation to adjust the range gate marker on a rendering image. That is, the configuration and the modified examples described in the above-described embodiments are applicable to the fifth embodiment except that the reception function 173 receives an operation to adjust the range gate marker on a rendering image.

### Sixth Embodiment

In the above-described embodiment, an explanation is given of a case where blood flow measurement is conducted once by using echography; however, the embodiment is applicable to a case where, for example, echography is individually conducted more than once. In this case, the range gate marker and the angle correction marker, used during the first ultrasound examination, may be used during the second and subsequent ultrasound examinations. Therefore, in the sixth embodiment, an explanation is given of a case where the range gate marker and the angle correction marker, used during the first ultrasound examination, may be used during the second and subsequent ultrasound examinations.

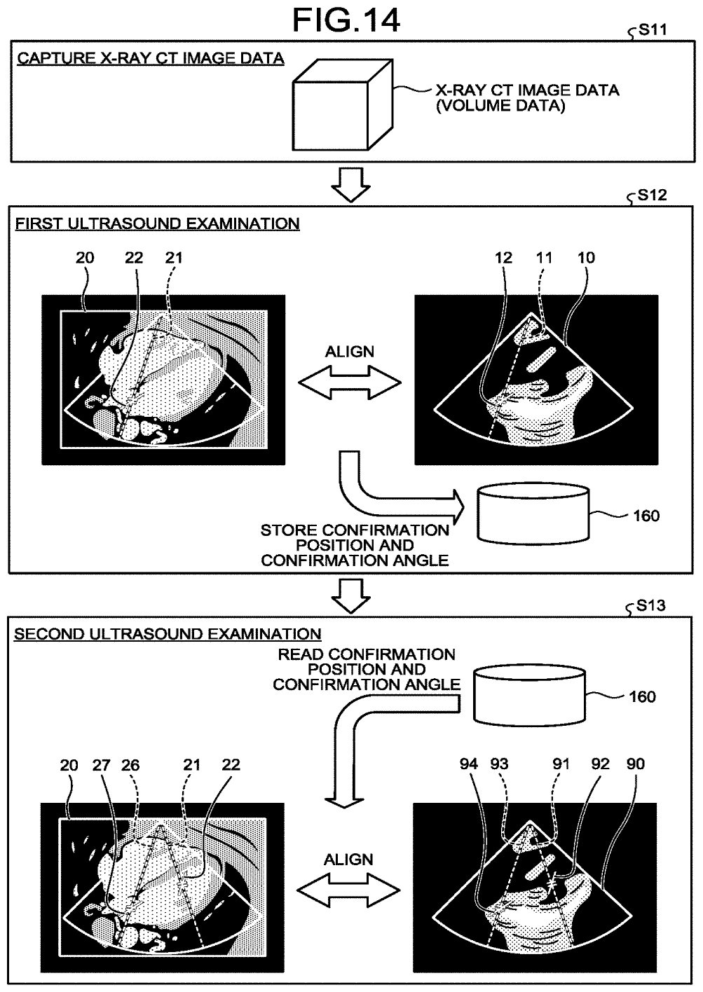

FIG. 14 is a diagram that illustrates a process of the ultrasonic diagnostic device 1 according to the sixth embodiment. FIG. 14 illustrates a case where X-ray CT image data capturing (S11), the first ultrasound examination (S12), and the second ultrasound examination (S13) are sequentially performed.

Here, examples of the case where echography is conducted multiple times as in FIG. 14 include a case where a coronary-artery stent placement operation is performed to expand a narrowed site of the coronary artery by using a stent. In this case, echography is conducted twice in total before and after the stent is placed so that a blood-flow improvement effect due to the coronary-artery stent placement operation is evaluated. Here, the coronary-artery stent placement operation is only an example, and this is not a limitation. The present embodiment may be widely applied to a case where blood-flow information at the same blood vessel position is evaluated 2 or more different times.

As illustrated in FIG. 14, at S11, capturing of X-ray CT image data is conducted. Here, capturing of X-ray CT image data may be conducted at any time before the first ultrasound examination. Capturing of X-ray CT image data may be conducted at any time, e.g., immediately before the first ultrasound examination, a few days earlier, or a few weeks earlier.

At S12, the first ultrasound examination is conducted. For example, the display control function 172 causes the ultrasonic image 10 and the 2D CT image 20 to be presented on the display 103 during the same process as that described in the first embodiment. Here, the ultrasonic image 10 is equivalent to the B-mode image captured during the first ultrasound examination at S12. Furthermore, the 2D CT image 20 is equivalent to the X-ray CT image data that is captured at S11. Furthermore, the display control function 172 causes the range gate marker 11 and the angle correction marker 12 to be presented on the ultrasonic image 10. Moreover, the display control function 172 causes the range gate marker 21 and the angle correction marker 22 to be presented on the 2D CT image 20.

Furthermore, due to the same process as that described in the first embodiment, the positions of the range gate marker 11 and the range gate marker 21 are in conjunction with each other. Moreover, due to the same process as that described in the first embodiment, the angles of the angle correction marker 12 and the angle correction marker 22 are in conjunction with each other. For this reason, for example, the operator may adjust the position of the range gate marker 11 and the angle of the angle correction marker 12 on the ultrasonic image 10 by adjusting the position of the range gate marker 21 and the angle of the angle correction marker 22 on the 2D CT image 20. Thus, the operator may adjust the range gate marker 11 and the angle correction marker 12 to the desired position and angle and collect blood-flow information during the first ultrasound examination.

Here, if a confirmation operation to confirm the position of the position marker on the display image is received from the operator, the reception function 173 according to the sixth embodiment further stores a confirmation position, which indicates the position of the position marker when the confirmation operation is performed, in the internal memory 160. Specifically, at S12, if the operator performs an operation (confirmation operation) to confirm the position of the range gate marker 21 on the 2D CT image 20, the reception function 173 stores the position of the range gate marker 21 at S12 as “confirmation position” in the internal memory 160.

Furthermore, if a confirmation operation is received from the operator, the reception function 173 according to the sixth embodiment further stores the confirmation angle, which indicates the angle of the angle marker when the confirmation operation is performed, in the internal memory 160. Specifically, at S12, if the operator performs an operation (confirmation operation) to confirm the angle of the angle correction marker 22 on the 2D CT image 20, the reception function 173 stores the angle of the angle correction marker 22 at S12 as a “confirmation angle” in the internal memory 160.

At S13, the second ultrasound examination is conducted. Here, the second ultrasound examination may be conducted at any time after the first ultrasound examination. For example, if the coronary-artery stent placement operation is performed, it is preferable that the second ultrasound examination is performed immediately after that; however, this is not a limitation. For example, if blood-flow information is evaluated on a regular basis, the second ultrasound examination may be conducted at any time, e.g., a few days later, a few weeks later, or a few months later.

For example, the display control function 172 causes an ultrasonic image 90 and the 2D CT image 20 to be presented on the display 103 during the same process as that described in the first embodiment. Here, the ultrasonic image 90 is equivalent to the B-mode image that is captured during the second ultrasound examination at S13. Furthermore, the 2D CT image 20 is equivalent to the X-ray CT image data that is captured at S11. Furthermore, the display control function 172 causes a range gate marker 91 and an angle correction marker 92 to be presented on the ultrasonic image 90. Furthermore, the display control function 172 causes the range gate marker 21 and the angle correction marker 22 to be presented on the 2D CT image 20.

Here, if new ultrasonic image data, which is different from the ultrasonic image data during the first ultrasound examination, is acquired, the display control function 172 according to the sixth embodiment further causes a new position marker based on the confirmation position to be displayed on the display image based on at least any one of the new ultrasonic image data and the volume data.

For example, the display control function 172 reads the confirmation position from the internal memory 160. The confirmation position is the information stored in the internal memory 160 at S12. Then, the display control function 172 causes a new range gate marker 93 based on the confirmation position to be presented on the ultrasonic image 90. Furthermore, the display control function 172 causes a new range gate marker 26 based on the confirmation position to be presented on the 2D CT image 20.

Specifically, the range gate marker 93 and the range gate marker 26 are markers that indicate the positions of the range gate markers 11 and 21, confirmed at S12 (the first ultrasound examination). For this reason, the operator may easily know the positions of the range gate markers during the previous ultrasound examination by only checking the positions of the range gate markers 93, 26. Therefore, at S13 (the second ultrasound examination), by adjusting the positions of the range gate markers 91, 21 so as to match the positions of the range gate markers 93, 26, the operator may easily match the current position of the range gate marker to the previous position of the range gate marker.

Furthermore, if new ultrasonic image data, which is different from the ultrasonic image data during the first ultrasound examination, is acquired, the display control function 172 according to the sixth embodiment further causes a new angle marker based on the confirmation angle to be displayed on the display image based on at least any one of the new ultrasonic image data and the volume data.

For example, the display control function 172 reads the confirmation angle from the internal memory 160. The confirmation angle is the information stored in the internal memory 160 at S12. Then, the display control function 172 causes a new angle correction marker 94 based on the confirmation angle to be presented on the ultrasonic image 90. Furthermore, the display control function 172 causes a new angle correction marker 27 based on the confirmation angle to be presented on the 2D CT image 20.

Specifically, the angle correction marker 94 and the angle correction marker 27 are markers that indicate the angles of the angle correction markers 12, 22, confirmed at S12 (the first ultrasound examination). For this reason, the operator may easily know the angles of the angle correction markers during the previous ultrasound examination by only checking the angles of the angle correction markers 94, 27. Therefore, the operator adjusts the angles of the angle correction markers 92, 22 at S13 (the second ultrasound examination) so as to match the angles of the angle correction markers 94, 27, whereby the current angle of the angle correction marker is easily matched with the previous angle of the angle correction marker.

In this way, the ultrasonic diagnostic device 1 according to the sixth embodiment may use the range gate marker and the angle correction marker, which are used during the first ultrasound examination, during the second ultrasound examination. Furthermore, although an explanation is given in FIG. 14 of a case where ultrasound examinations are performed twice, the same holds for a case where ultrasound examinations are performed three or more times. That is, if ultrasound examinations are performed three or more times, the ultrasonic diagnostic device 1 may use the range gate marker and the angle correction marker, which are used during the first ultrasound examination, during the third and subsequent ultrasound examinations.

Here, for the convenience of illustrations, FIG. 14 illustrates only the ultrasonic image and the 2D CT image; however, this is not a limitation on the embodiment. For example, as illustrated in FIG. 3A, or the like, the display control function 172 may present the Doppler waveform 30 or the measurement result 40 on the display 103.

Furthermore, in FIG. 14, an explanation is given of a case where the range gate marker and the angle correction marker, which have been confirmed, are presented; however, this is not a limitation on the embodiment. For example, the display control function 172 may cause the information for navigation to be displayed on the basis of the difference between the position of the confirmed range gate marker and the position of the currently set range gate marker. In this case, the display control function 172 may present the image that indicates the direction in which the range gate marker is to be adjusted (the image that is shaped like an arrow, or the like) or the information that indicates the amount of adjustment (the numerical value that indicates a distance, or the like). Furthermore, the display control function 172 may also present the information for navigation on the basis of a difference for the angle correction marker.

### Other Embodiments

Other than the above-described embodiments, various different embodiments may be implemented.

### (Application to the CWD Method)

For example, in the cases explained, the above-described embodiments and modified examples are applied to collection of blood-flow information (Doppler waveform) according to the PWD method; however, this is not a limitation on the embodiment. For example, the above-described embodiments and modified examples are applicable to collection of blood-flow information according to the CWD method. For example, in the CWD mode, the reception function 173 receives an operation to set the position marker, which indicates a linear sampling position, from the operator. Furthermore, the display control function 172 causes the position marker to be displayed at a corresponding position on the display image based on at least the volume data captured by a different medical-image diagnostic device in accordance with the correspondence relation.

(Simultaneous Display with Medical Image Data from a Different Medical-Image Diagnostic Device)

Furthermore, for example, in the above-described embodiments and modified examples, an explanation is given of a case where X-ray CT image data is applied as an example of the medical image data captured by a medical-image diagnostic device that is different from the ultrasonic diagnostic device 1; however, this is not a limitation on the embodiment. For example, the ultrasonic diagnostic device 1 is applicable to a case where MRI image data and B-mode image data are simultaneously displayed.

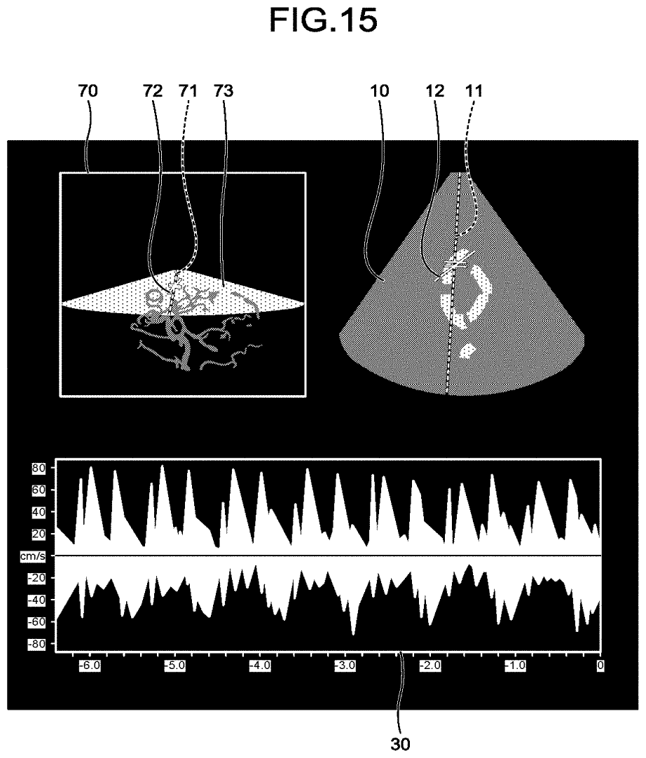

FIG. 15 is a diagram that illustrates a process of the display control function 172 according to a different embodiment. As illustrated in FIG. 15, the display control function 172 causes the ultrasonic image 10, an MRI image 70, and the Doppler waveform 30 to be presented. Here, as the Doppler waveform 30 is the same as that in FIG. 3A, its explanation is omitted.

For example, the display control function 172 presents the MRI image 70 that captures the area including the brain of the subject P. In the example illustrated in FIG. 15, the arterial circle of Willis is rendered on the MRI image 70. Furthermore, the display control function 172 causes a range gate marker 71, an angle correction marker 72, and a scan area marker 73 to be presented on the MRI image 70. Here, on the MRI image 70, the range gate marker 71 and the angle correction marker 72 are markers that correspond to the position of the range gate marker 11 and the angle of the angle correction marker 12. Furthermore, the scan area marker 73 is a frame border that indicates the position of the ultrasonic image 10 on the MRI image 70.

Furthermore, the display control function 172 presents the ultrasonic image 10, on which the brain of the subject P is rendered, together with the MRI image 70. The ultrasonic image 10 is captured when the ultrasonic probe 101 conducts ultrasonic scanning on the area that includes the brain of the subject P.

Thus, the above-described embodiments and modified examples are applicable to a case where the ultrasonic diagnostic device 1 simultaneously presents ultrasonic image data and medical image data other than X-ray CT image data.

(Two Time-Phases Display of Medical Image Data from a Different Medical-Image Diagnostic Device)

Furthermore, for example, in FIG. 11, an explanation is given of a case where pieces of ultrasonic image data in two different time phases are simultaneously displayed; however, this is not a limitation on the embodiment. For example, the ultrasonic diagnostic device 1 may display pieces of medical image data from a different medical-image diagnostic device, which is different from the ultrasonic diagnostic device 1, in two different time phases.

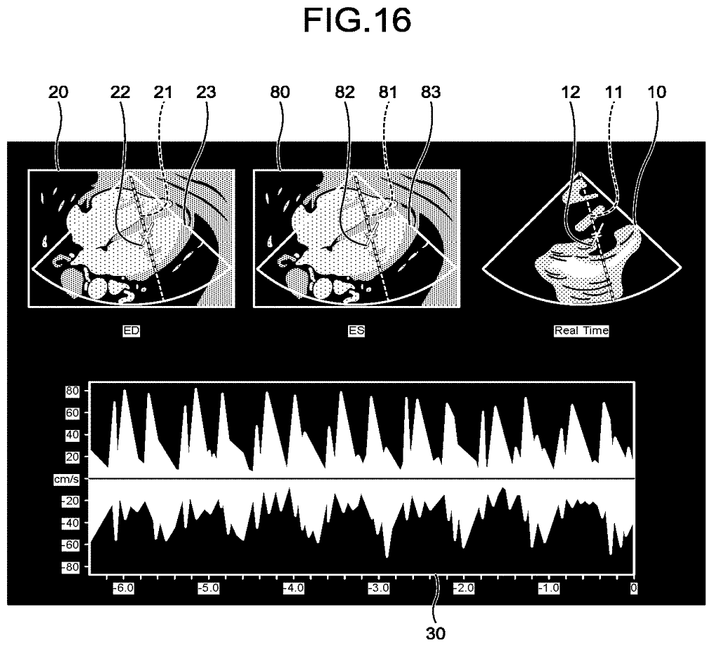

FIG. 16 is a diagram that illustrates a process of the display control function 172 according to a different embodiment. FIG. 16 illustrates an example of the display screen presented on the display 103 due to the process of the display control function 172. Furthermore, in FIG. 16, the X-ray CT image data is dynamic volume data (4D CT image data) that is obtained by capturing three-dimensional volume data multiple times at a predetermined frame rate (volume rate).

As illustrated in FIG. 16, the display control function 172 causes the 2D CT image 20 at the end diastole (ED) and a 2D CT image 80 at the end systole (ES) to be simultaneously displayed. Furthermore, as the ultrasonic image 10 and the Doppler waveform 30 are the same as those in FIG. 3A, their explanations are omitted.

In this manner, the display control function 172 causes the 2D CT images 20, 80 in two different time phases (two timings) to be displayed. Thus, the operator may select a 2D CT image in the time phase that is appropriate for adjustment of the range gate marker and the angle correction marker. For example, in the case of patients with tachycardia or arrhythmias, it is not always possible to specify an image at an appropriate timing. Furthermore, if images are significantly blurred, it is difficult to recognize an image at an appropriate timing. Therefore, the ultrasonic diagnostic device 1 causes the 2D CT images 20, 80 in two different time phases (two timings) to be presented so that the operator may select the 2D CT image in an appropriate time phase. For this reason, the operator may select a 2D CT image in an appropriate time phase even if a patient has tachycardia or arrhythmias or if an image is significantly blurred. Furthermore, for example, the operator holds a 2D CT image in the time phase, which is supposed to be appropriate, while causing a 2D CT image to be presented by switching the time phase manually or automatically, whereby a more appropriate time phase may be selected.

Here, the contents illustrated in FIG. 16 are only an example, and the illustrated contents are not a limitation. For example, the contents illustrated in FIG. 16 may be implemented by being combined with the case (FIG. 11) where pieces of ultrasonic image data in two different time phases are simultaneously displayed.

### (Medical-Image Processing Device)

For example, in the embodiments and the modified examples that are described above, an explanation is given of a case where the ultrasonic diagnostic device 1 performs the respective processing functions, implemented by the acquisition function 171, the display control function 172, and the reception function 173 that are components of the processing circuitry 170; however, this is not a limitation on the embodiment. For example, each of the above-described processing functions may be performed by a medical-image processing device, such as workstation. Furthermore, in this case, the acquisition function 171 may acquire the positional information that is previously stored in relation to ultrasonic image data instead of acquiring the positional information on ultrasonic image data from the position detection system. Furthermore, if the correspondence relation between a position in the ultrasonic image data and a position in the volume data, captured by a different medical-image diagnostic device that is different from the ultrasonic diagnostic device 1, is already generated and stored in a predetermined memory circuit, the acquisition function 171 may acquire the correspondence relation.

Furthermore, components of each device illustrated are functionally conceptual and do not necessarily need to be physically configured as illustrated in the drawings. Specifically, specific forms of separation and combination of each device are not limited to those depicted in the drawings, and a configuration may be such that all or some of them are functionally or physically separated or combined in an arbitrary unit depending on various types of loads, usage, or the like. Furthermore, all or any of various processing functions performed by each device may be implemented by a CPU and programs analyzed and executed by the CPU or may be implemented as wired logic hardware.

Furthermore, among the processes described in the above embodiments and modified examples, all or some of the processes that are automatically performed as described may be performed manually, or all or some of the processes that are manually performed as described may be performed automatically by using a well-known method. Furthermore, the operation procedures, the control procedures, the specific names, and the information including various types of data and parameters as described in the above specifications and the drawings may be arbitrarily changed except as otherwise noted.

Furthermore, the image processing method explained in the above embodiments and modified examples may be implemented when a prepared image processing program is executed by a computer, such as a personal computer or workstation. The image processing method may be distributed via a network, such as the Internet. Furthermore, the ultrasonic imaging method may be recorded in a recording medium readable by computers, such as a hard disk, flexible disk (FD), CD-ROM, MO, or DVD, and read from the recording medium by the computer to be executed.

Furthermore, in the above-described embodiments and modified examples, substantially in real time means that each process is performed immediately each time each piece of data, which is the target to be processed, is generated. For example, the process to display an image substantially in real time is the idea that includes not only a case where the time when the subject is captured completely matches the time when the image is displayed, but also a case where the image is displayed with a slight delay due to the time required for each process, such as image processing.

Furthermore, in the above-described embodiments and modified examples, the substantially identical cardiac time phase is the idea that includes not only the cardiac time phase that completely matches a certain cardiac time phase, but also the cardiac time phase that is shifted without having any effects on the embodiment or the cardiac time phase that is shifted due to a detection error of an electrocardiographic waveform. For example, if a B-mode image in a desired cardiac time phase (e.g., the R wave) is obtained, there are sometimes no B-mode images that completely match the R wave in accordance with a frame rate of the ultrasonic diagnostic device 1. In this case, an interpolation process is performed by using B-mode images in the frames before and after the R wave so that the B-mode image, which is supposed to be the R wave, may be generated, or the B-mode image in the time close to the R wave may be selected as a B-mode image of the R wave. Furthermore, the B-mode image selected here is preferably the one closest to the R wave; however, the one that is not closest is selectable without having any effects on the embodiment.

According to at least one of the above-described embodiments, the accuracy and the quantitative characteristic of blood-flow information may be improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic device, comprising:
an ultrasonic probe configured to conduct ultrasonic scanning on a three-dimensional area of a subject and receive a reflected wave from the subject; and
processing circuitry configured to
acquire a correspondence relation between a position in ultrasonic image data on the three-dimensional area based on the reflected wave and a position in volume data on the subject captured by a different medical-image diagnostic device;
receive, from an operator, a operation that sets a first angle marker for conducting angle correction in blood-flow information, on a scan area of the ultrasonic image data in a first display image;
set the first angle marker based on the operation;
cause a second angle marker to be displayed at an angle corresponding to a position on a second display image based on the volume data, in accordance with a position and an angle of the first angle marker on the scan area of the ultrasonic image data in the first display image and the correspondence relation; and
adjust, based on the operation, the angle of the first angle marker on the scan area of the ultrasonic image data in the first display image in conjunction with the angle of the second angle marker in the second display image, based on the volume data.

2. The ultrasonic diagnostic device according to claim 1, wherein the processing circuitry is further configured to:
acquire a cardiac time phase of the subject; and
in accordance with the cardiac time phase, cause an ultrasonic image in a cardiac time phase that is substantially identical to a cardiac time phase in the volume data to be displayed as the first display image.

3. The ultrasonic diagnostic device according to claim 1, wherein the processing circuitry is further configured to:
cause a first position marker, which indicates a position at which blood-flow information is extracted on a scan area of the ultrasonic image data, to be displayed on the first display image; and
cause a second position marker at a corresponding position on the second display image in accordance with the position of the first position marker and the correspondence relation.

4. The ultrasonic diagnostic device according to claim 1, wherein the processing circuitry is further configured to
receive an angle change operation to change an angle of the second angle marker on the third second display image, and
change the angle of the angle marker in accordance with the angle change operation.

5. The ultrasonic diagnostic device according to claim 1, wherein each time the angle of the angle marker is changed, the processing circuitry causes a measurement value of the blood-flow information, whose angle has been corrected at the changed angle, to be displayed.

6. The ultrasonic diagnostic device according to claim 1, wherein the processing circuitry is further configured to calculate an index value related to the subject by using a first measurement value, measured from the ultrasonic image data or the blood-flow information, and a second measurement value, measured from the volume data.

7. The ultrasonic diagnostic device according to claim 1, wherein the processing circuitry is further configured to
cause a first cross-sectional image, which corresponds to a scan cross-sectional surface on which the ultrasonic scanning is conducted, to be displayed as the first display image, and
cause a second cross-sectional image at a position that corresponds to the first cross-sectional image to be displayed as the second display image based on the volume data.

8. The ultrasonic diagnostic device according to claim 7, wherein the processing circuitry is further configured to
cause a rendering image, generated during a rendering process on the volume data, to be displayed as the second display image, and
cause a cross-sectional position that corresponds to the first cross-sectional image and a cross-sectional position that corresponds to the second cross-sectional image to be displayed on the rendering image.

9. The ultrasonic diagnostic device according to claim 1, wherein
the ultrasonic probe conducts ultrasonic scanning on an area that includes coronary artery of the subject, and
the processing circuitry causes an ultrasonic image, on which the coronary artery is rendered, to be displayed.

10. The ultrasonic diagnostic device according to claim 2, wherein the processing circuitry causes an ultrasonic image generated substantially in real time to be displayed separately from an ultrasonic image in a cardiac time phase that is substantially identical to a cardiac time phase in the volume data.

11. The ultrasonic diagnostic device according to claim 1, wherein
the ultrasonic probe conducts ultrasonic scanning on an area that includes a brain of the subject, and
the processing circuitry causes an ultrasonic image, on which the brain is rendered, to be displayed together with the third display image.

12. The ultrasonic diagnostic device according to claim 1, wherein the processing circuitry causes a third display image, which is based on volume data captured in a first time phase, and a fourth display image, which is based on volume data captured in a second time phase that is different from the first time phase, to be simultaneously displayed as the second display image.

13. The ultrasonic diagnostic device according to claim 1, wherein the processing circuitry is further configured to
When a confirmation operation for confirming an angle of the second angle marker is received from an operator, store a confirmation angle, which indicates an angle of the second angle marker when the confirmation operation is performed, in a memory, and
when new ultrasonic image data, which is different from the ultrasonic image data, is acquired, cause a new angle marker based on the confirmation angle to be displayed on a new display image that is based on at least any one of the new ultrasonic image data and the volume data.

14. An image processing device, comprising:
processing circuitry configured to
acquire a correspondence relation between a position in ultrasonic image data on a three-dimensional area of a subject, which is based on a reflected wave received from the three-dimensional area by using a ultrasonic probe, and a position in volume data on the subject captured by a different medical-image diagnostic device that is different from an ultrasonic diagnostic device;
receive, from an operator, a operation that sets a first angle marker for conducting angle correction in blood-flow information, on a scan area of the ultrasonic image data in a first display image;
set the first angle marker based on the operation;
cause a second angle marker to be displayed at an angle corresponding to a position on a second display image based on the volume data in accordance with a position and an angle of the first angle marker on the scan area of the ultrasonic image data in the first display image and the correspondence relation; and
adjust, based on the operation, the angle of the first angle marker on the scan area of the ultrasonic image data in the first display image in conjunction with the angle of the second angle marker in the second display image based on the volume data.

15. An image processing method, comprising:
acquiring a correspondence relation between a position in ultrasonic image data on a three-dimensional area of a subject, which is based on a reflected wave received from the three-dimensional area by using a ultrasonic probe, and a position in volume data on the subject captured by a different medical-image diagnostic device that is different from an ultrasonic diagnostic device;
receiving, from an operator, a operation that sets a first position angle marker for conducting angle correction in blood-flow information, on a scan area of the ultrasonic image data in a first display image;
setting the first angle marker based on the operation;
causing a second angle marker to be displayed at an angle corresponding to a position on a display image based on the volume data in accordance with a position and an angle of the first angle marker on the scan area of the ultrasonic image data in the first display image and the correspondence relation; and
adjusting, based on the operation, the angle of the first angle marker on the scan area of the ultrasonic image data in the first display image in conjunction with the angle of the second angle marker in the second display image based on the volume data.
